SEQUENCE LISTING (22 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 269 aa, Artificial Sequence
  BLAP 101E
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - protein, 269 aa, Artificial Sequence
  BLAP 3T, 4I, 101E, 205I
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR

SEQ ID NO: 5 - protein, 269 aa, Artificial Sequence
  BLAP variant 3T, 4I, 205I
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR

SEQ ID NO: 6 - protein, 269 aa, Artificial Sequence
  variant of Thermomyces lanuginosus lipase
EVSQDLFNQFKLFAQYSKAAYCKANNDAPAGTNITCTGNACPEVEKADATFLYSFEDSGVGDVTGFLALDNTNKLIILSFRGSRSIENWIGNLNFDLKEINDICSGCRGHDGFTSSWRSVADTLRQKVEAAVREHPDYRVVFTGHSLGGALATIAGADLRGNGYDIDVFSYGAPRVGNRAFAEFLTTQQGGTLYRITHTNDIVPRLPPREFGYSHSSPEYWIKSGTLVPVTRNDIVKIEGIDATGGNNQPNIPDIPAHLWYFGLIGTCL

SEQ ID NO: 7 - protein, 269 aa, Artificial Sequence
  variant of Thermomyces lanuginosus lipase
EVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 8 - protein, 7 aa, Artificial Sequence
  loop sequence
ADGEGAI

SEQ ID NO: 9 - protein, 7 aa, Artificial Sequence
  loop sequence
ADGDGAI

SEQ ID NO: 10 - protein, 7 aa, Artificial Sequence
  loop sequence
ADGDGSV

SEQ ID NO: 11 - protein, 7 aa, Artificial Sequence
  loop sequence
ADGEGSV

SEQ ID NO: 12 - protein, 8 aa, Artificial Sequence
  loop sequence
AADGEGSV

SEQ ID NO: 13 - protein, 8 aa, Artificial Sequence
  loop sequence
ASEGEGSV

SEQ ID NO: 14 - protein, 4 aa, Artificial Sequence
  peptide
LGAY

SEQ ID NO: 15 - protein, 4 aa, Artificial Sequence
  peptide
FGAY

SEQ ID NO: 16 - protein, 4 aa, Artificial Sequence
  peptide
YGAY

SEQ ID NO: 17 - protein, 4 aa, Artificial Sequence
  peptide
FGVY

SEQ ID NO: 18 - protein, 4 aa, Artificial Sequence
  peptide
FGAM

SEQ ID NO: 19 - protein, 4 aa, Artificial Sequence
  peptide
WLVY

SEQ ID NO: 20 - protein, 4 aa, Artificial Sequence
  peptide
FGAL

SEQ ID NO: 21 - protein, 4 aa, Artificial Sequence
  peptide
FGAF

SEQ ID NO: 22 - protein, 4 aa, Artificial Sequence
  peptide
LGAL

(19) United States
(12) Patent Application Publication
(10) Pub. No.: US 2024/0240114 A1
(43) Pub. Date: Jul. 18, 2024

(54) Improved Combination of Protease and Protease Inhibitor with Secondary Enzyme

(71) Applicant: BASF SE, Ludwigshafen (DE)

(72) Inventors: Stefan Jenewein, Ludwigshafen (DE); Gabriele Boenemann, Basel (CH)

(21) Appl. No.: 18/027,461

(22) PCT Filed: Sep. 17, 2021
(86) PCT No.: PCT/EP2021/075665

§ 371 Date: Mar. 21, 2023

(30) Foreign Application Priority Data
Sep. 22, 2020 (EP) 20197482.1
Oct. 9, 2020 (EP) 20201088.0

(51) Int. Cl. C11D 3/386; C11D 3/32; C12N 9/54

(52) U.S. Cl. CPC C11D 3/386; C11D 3/32; C12N 9/54

(57) ABSTRACT

The present invention relates to a composition comprising an improved combination of protease and protease inhibitor. It was revealed that the combination of a protease with introduced negative charges in the active site loop and a peptide aldehyde-based protease inhibitor results in an increased stability of additional enzymes being also present in a composition together with the protease and the peptide aldehyde-based protease inhibitor. This is in particular beneficial for detergent compositions comprising proteases together with other enzymes and which frequently suffer from enzyme instability during storage.

### FIELD OF THE INVENTION

The present invention relates to a composition comprising an improved combination of protease and protease inhibitor. It was revealed that the combination of a protease with introduced negative charges in the active site loop and a peptide aldehyde-based protease inhibitor results in an increased stability of additional enzymes being also present in a composition together with the protease and the peptide aldehyde-based protease inhibitor. This is in particular beneficial for detergent compositions comprising proteases together with other enzymes and which frequently suffer from enzyme instability during storage.

### BACKGROUND OF THE INVENTION

Enzymes are increasingly used in various application as sustainable alternative to petrochemistry. Enzymes are biodegradable and can be catalytically active already at lower temperatures, which results in reduction of energy consumption. An attempt to improve cleaning efficiency and thereby reducing energy consumption in a washing step is the use of enzymes in detergent compositions. Particularly, proteases are nowadays routinely used in detergent compositions. However, the use of enzymes is hampered by the instability of these biomolecules, in particular, in detergent compositions. Moreover, proteases degrade themselves and also other enzymes present in the formulation. To overcome this deficiency, protease inhibitors can be use. One type of protease are peptide aldehydes and peptide aldehyde hydrosulfite adducts.

WO 2009/118375 disclose detergents with a subtilisin-type proteases stabilized by a peptide aldehyde. WO 2013/004636 discloses a composition comprising a subtilisin and a peptide aldehyde hydrosulfite adduct.

Subtilisins are a class of proteases widely used in commercial products (for example, in laundry and dish washing detergents, and contact lens cleaners) and for research purposes (catalysts in synthetic organic chemistry). Various attempts have been made to modify the amino acid sequence of subtilisins in order to improve the biochemical properties of these enzymes, in particular with respect to their stability and wash performance. One member of the subtilisin family, a highly alkaline protease from Bacillus lentus, and its variants for use in detergent formulations has been described in patent application WO9102792 (BLAP, SEQ ID NO: 1).

However, there is still a need for an improvement of enzyme stability in compositions, in particular detergent compositions, comprising a peptide stabilizer. Especially when proteases are present there is a need to efficiently stabilize secondary enzymes added to the protease containing formulations.

The present inventors identified a that the use of a particular subtilisin protease improves the stability of secondary enzymes also present a composition comprising a protease inhibitor.

### BRIEF SUMMARY OF THE INVENTION

The present invention is directed to a composition comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

Moreover, the present invention is directed to a detergent composition comprising the composition of the present invention, in particular a detergent composition suitable for dish washing (preferably automated dishwashing (ADW)) and laundry.

Furthermore, the present invention is directed to a method for providing a detergent composition with improved stability and/or wash performance of an enzyme in the detergent composition, wherein the enzyme is not a protease, comprising the use of a detergent composition of the present invention.

Furthermore, the present invention is directed to the use of a composition of the present invention for providing an improved stability and/or wash performance of an enzyme in a detergent composition, wherein the enzyme is not a protease.

### DETAILED DESCRIPTION OF THE INVENTION

The present invention may be understood more readily by reference to the following detailed description of the embodiments of the invention and the examples included herein.

Although the present invention will be described with respect to particular embodiments, this description is not to be construed in a limiting sense.

### Definitions

Unless otherwise noted, the terms used herein are to be understood according to conventional usage by those of ordinary skill in the relevant art.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all of these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

The term “introduction of at least two negative charges” into a particular amino acid sequence refers to the increase of the net charge of the particular amino acid sequence by at least two negative charges. Such increase of the net charge of the particular amino acid sequence by at least two negative charges is achieved by altering the amino acid sequence and can be reached by one or more amino acid sequence alterations selected from the group consisting of substitution, deletion and insertion, preferably by one or more amino acid substitutions. The increase of the net charge of the particular amino acid sequence by at least two negative charges can be achieved by removing positive charges or by introducing negative charges or by combinations thereof. The four amino acids aspartic acid (Asp, D), glutamic acid (Glu, E), lysine (Lys, K), and arginine (Arg, R) have a side chain which can be charged at neutral pH. At pH 7.0, two are negatively charged: aspartic acid (Asp, D) and glutamic acid (Glu, E) (acidic side chains), and two are positively charged: lysine (Lys, K) and arginine (Arg, R) (basic side chains). Thus, the introduction of at least two negative charges in the amino acid sequence can be reached for instance by substituting arginine by glutamic acid, substituting two non-charged leucine residues by two glutamic acid residues, by inserting two aspartic acid residues or by deleting two lysine residues. The introduction of at least two negative charges by modification of the amino acid sequence is evaluated preferably under conditions usually occurring in a washing step, preferably at pH 6-11, preferably at pH 7-9, more preferably at pH 7.5-8.5, further preferred at pH 7.0-8.0, most preferably at pH 7.0 or pH 8.0.

“Parent” sequence (also called “parent enzyme” or “parent protein”) is the starting sequences for introduction of changes (e.g. by introducing one or more amino acid substitutions) of the sequence resulting in “variants” of the parent sequences. Thus, the term “enzyme variant” or “sequence variant” or “protein variant” are used in reference to parent enzymes that are the origin for the respective variant enzymes. Therefore, parent enzymes include wild type enzymes and variants of wild-type enzymes which are used for development of further variants. Variant enzymes differ from parent enzymes in their amino acid sequence to a certain extent; however, variants at least maintain the enzyme properties of the respective parent enzyme. Preferably, enzyme properties are improved in variant enzymes when compared to the respective parent enzyme. More preferably, variant enzymes have at least the same enzymatic activity when compared to the respective parent enzyme or variant enzymes have increased enzymatic activity when compared to the respective parent enzyme.

In describing the variants of the present invention, the abbreviations for single amino acids used according to the accepted IUPAC single letter or three letter amino acid abbreviation is used.

“Substitutions” are described by providing the original amino acid followed by the number of the position within the amino acid sequence, followed by the substituted amino acid. For example the substitution of histidine at position 120 with alanine is designated as “His120Ala” or “H120A”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as e.g. a Lys and Ala after Gly180 this may be indicated as: Gly180GlyLysAla or G195GKA.

In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG. Variants comprising multiple alterations are separated by “+”, e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma e.g. R170Y G195E or R170Y, G195E respectively. Where different alterations can be introduced at a position, the different alterations are separated by a comma, e.g. “Arg170Tyr, Glu” and R170T, E, respectively, represents a substitution of arginine at position 170 with tyrosine or glutamic acid. Alternatively, different alterations or optional substitutions may be indicated in brackets, e.g., Arg170[Tyr, Gly] or Arg170{Tyr, Gly} or in short R170 [Y, G] or R170 {Y, G}.

The numbering of the amino acid residues of the subtilisin proteases described herein is as commonly used for subtilisin proteases (cf. P. N. Bryan, Biochimica et Biophysica Acta 1543 (2000), 203-222, cf. p. 204, left col., 3^{rd} para.) according to the numbering of the BPN′ subtilisin protease from Bacillus amyloliquefaciens as shown in SEQ ID NO: 2 (i.e., according to the numbering of SEQ ID NO: 2 or according to “BPN′ numbering”).

Variants of the parent enzyme molecules may have an amino acid sequence which is at least n percent identical to the amino acid sequence of the respective parent enzyme having enzymatic activity with n being an integer between 50 and 100, preferably 50, 55, 60, 65, 70, 75, 80, 85, 90, 91, 92, 93, 94, 95, 96, 97, 98 or 99 compared to the full length polypeptide sequence. Preferably, variant enzymes which are n percent identical when compared to a parent enzyme, have enzymatic activity.

“Identity” in relation to comparison of two amino acid sequences herein is calculated by dividing the number of identical residues by the length of the alignment region which is showing the shorter sequence over its complete length. This value is multiplied with 100 to give “percent-identity”. In an alternative embodiment, “Identity” in relation to comparison of two amino acid sequences herein is calculated by dividing the number of identical residues by the length of the alignment region which is showing the sequence of this invention over its complete length. This value is multiplied with 100 to give alternatively “percent-identity”.

To determine the percent-identity between two amino acid sequences (i.e. pairwise sequence alignment), two sequences have to be aligned over their complete length (i.e. global alignment) in a first step. For producing a global alignment of two sequences, any suitable computer program, like program “NEEDLE” (The European Molecular Biology Open Software Suite (EMBOSS)), program “MATGAT” (Campanella, J. J, Bitincka, L. and Smalley, J. (2003), BMC Bioinformatics, 4:29), program “CLUSTAL” (Higgins, D. G. and Sharp, P. M. (1988), Gene, 73, 237-244) or similar programs may be used. In lack of any program, sequences may also be aligned manually.

After aligning two sequences, in a second step, an identity value shall be determined from the alignment. Depending on the applied method for percent-identity calculation, different percent-identity values can be calculated from a given alignment. Consequently, computer programs which create a sequence alignment, and in addition calculate percent-identity values from the alignment, may also report different percent-identity values from a given alignment, depending which calculation method is used by the program. Therefore, the following calculation of percent-identity according to the invention applies:

percent-identity=(identical residues/length of the alignment region which is showing the shorter sequence over its complete length)*100.

In an alternative embodiment, the following calculation of percent-identity according to the invention applies:

percent-identity=(identical residues/length of the alignment region which is showing the sequence of this invention over its complete length)*100.

A special aspect concerning amino acid substitutions are conservative mutations which often appear to have a minimal effect on protein folding resulting in substantially maintained enzyme properties of the respective enzyme variant compared to the enzyme properties of the parent enzyme. Conservative mutations are those where one amino acid is exchanged with a similar amino acid. Such an exchange most probably does not change enzyme properties. Herein and in particular for the determination of percent-similarity the following conservative exchanges are considered:
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. Preferably, such mutations are not pertaining the functional domains of an enzyme, more preferably conservative mutations are not pertaining the catalytic centers of an enzyme.

To take conservative mutations into account, a value for “similarity” of two amino acid sequences may be calculated. “Similarity” in relation to comparison of two amino acid sequences herein is calculated by dividing the number of identical residues plus the number of similar residues by the length of the alignment region which is showing the shorter sequence over its complete length. This value is multiplied with 100 to give “percent-similarity”. In an alternative embodiment, “similarity” in relation to comparison of two amino acid sequences herein is calculated by dividing the number of identical residues plus the number of similar residues by the length of the alignment region which is showing the sequence of this invention over its complete length. This value is multiplied with 100 to alternatively give “percent-similarity”.

Therefore, the following calculation of percent-similarity according to the invention applies:

percent-similarity=[(identical residues+similar residues)/length of the alignment region which is showing the shorter sequence over its complete length]*100.

In an alternative embodiment, the following calculation of percent-similarity according to the invention applies:

percent-similarity=[(identical residues+similar residues)/length of the alignment region which is showing the sequence of this invention over its complete length]*100.

Especially, variant enzymes comprising conservative mutations which are at least m percent similar to the respective parent sequences with m being an integer between 50 and 100, preferably 50, 55, 60, 65, 70, 75, 80, 85, 90, 91, 92, 93, 94, 95, 96, 97, 98 or 99 compared to the full length polypeptide sequence, are expected to have essentially unchanged enzyme properties. Preferably, variant enzymes with m percent-similarity when compared to a parent enzyme, have enzymatic activity.

“Enzyme properties” include, but are not limited to catalytic activity as such, substrate/cofactor specificity, product specificity, increased stability in the course of time, thermostability, pH stability, chemical stability, and improved stability under storage conditions.

The term “substrate specificity” reflects the range of substrates that can be catalytically converted by an enzyme.

“Enzymatic activity” means the catalytic effect exerted by an enzyme, expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, etc.

The term “protease” (or alternatively “peptidase” or “proteinase”) is used for an enzyme with proteolytic activity, i.e., an enzyme that hydrolyses peptide bonds that link amino acids together in a polypeptide chain.

Enzymatic activity might change during storage or operational use of the enzyme. The term “enzyme stability” according to the current invention relates to the retention of enzymatic activity as a function of time during storage or operation. Retention of enzymatic activity as a function of time during storage is called “storage stability” and is preferred within the context of the invention.

To determine and quantify changes in catalytic activity of enzymes stored or used under certain conditions over time, the “initial enzymatic activity” is measured under defined conditions at time zero (100%) and at a certain point in time later (x %). By comparison of the values measured, a potential loss of enzymatic activity can be determined in its extent. The extent of enzymatic activity loss determines an enzyme's stability or non-stability.

“Half-life of enzymatic activity” is a measure for time required for the decaying of enzymatic activity to fall to one half (50%) of its initial value.

“Enzyme inhibitors” slow down the enzymatic activity by several mechanism as outlined below. Inhibitor binding is either reversible or irreversible. Irreversible inhibitors usually bind covalently to an enzyme by modifying the key amino acids necessary for enzymatic activity. Reversible inhibitors usually bind non-covalently (hydrogen bonds, hydrophobic interactions, ionic bonds).

Four general kinds of reversible inhibitors are known:
(1) substrate and inhibitor compete for access to the enzymes active site (competitive inhibition),
(2) inhibitor binds to substrate-enzyme complex (uncompetitive inhibition),
(3) binding of inhibitor reduces enzymatic activity but does not affect binding of substrate (noncompetitive inhibition),
(4) inhibitor can bind to enzyme at the same time as substrate (mixed inhibition).

For the purpose of the present invention, compounds comprising a stereocenter are considered to encompass and disclose both enantiomers, unless specifically indicated. In case that a compound comprises more than one stereocenter, all diastereomers as well as enantiomers are considered to be encompassed and disclosed, unless specifically indicated. If reference is made to a composition or mixture comprising a compound according to the present invention, it is understood that the compound can be present either as enantiomerically and/or diastereomerically pure compound or as a mixture of enantiomers and/or diastereomers, for example as a racemic mixture of the L or D-enantiomers of the amino acid residues as defined hereinafter. The same applies with regard to the synthesis of the compounds of the present invention, which compounds can be obtained either as enantiomerically and/or diastereomerically pure compounds or as a mixture of enantiomers and/or diastereomers, for example as a racemic mixture of the L or D-enantiomers of the amino acid residue as defined hereinafter.

As used herein, “wash performance” (also called herein “cleaning performance”) of an enzyme refers to the contribution of the enzyme to the cleaning performance of a detergent composition, i.e. the cleaning performance added to the detergent composition by the performance of the enzyme. The term “wash performance” is used herein similarly for laundry and hard surface cleaning. Wash performance is compared under relevant washing conditions. The term “relevant washing conditions” is used herein to indicate the conditions, particularly washing temperature, time, washing mechanics, sud concentration, type of detergent and water hardness, actually used in households in a dish detergent market segment. The term “improved wash performance” is used to indicate that a better end result is obtained in stain removal under relevant washing conditions, or that less enzyme, on weight basis, is needed to obtain the same end result relative to the corresponding control conditions.

As used herein, the term “specific performance” refers to the cleaning of specific stains or soils per unit of active enzyme. In some embodiments, the specific performance is determined using stains or sails such as egg, egg yolk, milk, grass, minced meat blood, chocolate sauce, baby food, sebum, etc.

A detergent composition and/or detergent solution of the invention comprises one or more detergent components. The term “detergent component” is defined herein to mean the types of chemicals, which can be used in detergent compositions and/or detergent solutions.

Detergent compositions and/or detergent solutions according to the invention include detergent compositions and/or detergent solutions for different applications such as laundry and hard surface cleaning.

The term “laundry” relates to both household laundering and industrial laundering and means the process of treating textiles and/or fabrics with a solution containing a detergent composition of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics made of these materials such as garments, cloths and other articles. The terms “fabric” (a textile made by weaving, knitting or felting fibers) or “garment” (any article of clothing made of textile) as used herein, mean to include the broader term textile as well.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheeps wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, wovens, or nonwovens.

The term “hard surface cleaning” is defined herein as cleaning of hard surfaces wherein hard surfaces may include any hard surfaces in the household, such as floors, furnishing, walls, sanitary ceramics, glass, metallic surfaces including cutlery or dishes. A particular form of hard surface cleaning is automatic dishwashing (ADW).

The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

In the technical field of the present invention, usually the term “stains” is used with reference to laundry, e.g., cleaning for textiles, fabric, or fibers, whereas the term “soils” is usually used with reference to hard surface cleaning, e.g., cleaning of dishes and cutlery. However, herein the terms “stain” and “soil” shall be used interchangeably.

The term “pilling” in this respect is the formation of pills and fuzz on the surface of cotton containing fabrics due to broken or disordered fibers.

The term “antipilling” is used to describe the prevention of the formation of pills and fuzz on the surface of cotton containing fabrics as well as the removal of pills and fuzz from cotton containing fabrics. Antipilling normally results in color clarification when colored cotton containing fabrics are treated.

The term “color clarification” in this respect is the reestablishment of the attractive fresh look of colored fabrics containing or consisting of cellulose based fibers, which have developed a greyish appearance by treatment, especially with laundry detergents, of the colored fabric. The term “redeposition” in this respect is deposition of dirt or color components that were removed from these textiles or fabrics during a laundry washing or textile treatment.

The term “anti-redeposition” in this respect is the action of cellulase to prevent or diminish the redeposition of dirt and color components on the fabric. Anti-redeposition may be called antigreying herein.

### DETAILED DESCRIPTION

### Protease

The composition according to the present invention comprise a protease as described herein. Also, the methods of the present invention comprise the use of a protease as described herein. The protease is a variant protease of the parent protease shown in SEQ ID NO: 1, preferably a subitilisin protease. Preferably, the variant protease comprises an amino acid sequence which is at least 80% identical to SEQ ID NO: 1 and wherein the amino acid sequence of the protease comprises compared to SEQ ID NO: 1 at least two additional negative charges in the loop region of residues 98 to 104 according to the numbering of SEQ ID NO: 2 (BPN′ numbering, i.e., wherein the positions are numbered by their correspondence to the amino acid sequence of subtilisin BPN′ of B. amyloliquefaciens, established as SEQ ID NO: 2), wherein preferably, the protease comprising as a catalytic triad the amino acids aspartate, histidine and serine, preferably the protease is a subitilisin protease.

Preferably, the protease described herein which comprises an amino acid sequence which is at least 80% identical to SEQ ID NO: 1 and wherein the amino acid sequence of the protease comprises compared to SEQ ID NO: 1 at least two additional negative charges in the loop region of residues 98 to 104 according to the numbering of SEQ ID NO: 2 shows an increased resistance against natural protease inhibitors, preferably against inhibitors comprised in stains, preferably in stains on textiles or hard surfaces, compared to a protease comprising an amino acid sequence which is at least 80% identical to SEQ ID NO: 1 and which does not comprise least two additional negative charges in the loop region of residues 98 to 104 according to the numbering of SEQ ID NO: 2, preferably compared to a protease comprising the amino acid sequence shown in SEQ ID NO: 1. Preferably, the protease shows reduced binding affinity to the naturally occurring protease inhibitors. Preferably the protease described herein is a subtilisin protease.

Preferably, the protease has at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 1 and comprises compared to SEQ ID NO: 1 at least two additional negative charges in the loop region of residues 98 to 104 according to the numbering of SEQ ID NO: 2. Preferably, the protease has at least 80%, at least 85%, at least 90%, at least 95%, at least 96%, at least 97%, at least 98%, at least 99%, or 100% sequence identity to SEQ ID NO: 1 and comprises compared to SEQ ID NO: 1 at least two additional negative charges in the loop region of residues 98 to 104 according to the numbering of SEQ ID NO: 2.

Preferably, the protease has at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 1 and comprises compared to SEQ ID NO: 1 the amino acid substitution R101D or R101E, preferably R101E, according to the numbering of SEQ ID NO: 2. Preferably, the protease has at least 80%, at least 85%, at least 90%, at least 95%, at least 96%, at least 97%, at least 98%, at least 99%, or 100% sequence identity to SEQ ID NO: 1 and comprises compared to SEQ ID NO: 1 the amino acid substitution R101D or R101E, preferably R101E, according to the numbering of SEQ ID NO: 2. Preferably, the protease has 100% sequence identity to SEQ ID NO: 1 and comprises compared to SEQ ID NO: 1 the amino acid substitution R101D or R101E, preferably R101E, according to the numbering of SEQ ID NO: 2, i.e., the protease comprises compared to SEQ ID NO: 1 only one amino acid exchange.

Preferably, the protease has at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 1 and comprises compared to SEQ ID NO: 1 the amino acid substitution R101D or R101E and the amino acid substitutions S3T, V4I, and V205I according to the numbering of SEQ ID NO: 2. More preferably, the protease has at least 80%, at least 85%, at least 90%, at least 95%, at least 96%, at least 97%, at least 98%, at least 99%, or 100% sequence identity to SEQ ID NO: 1 and comprises compared to SEQ ID NO: 1 the amino acid substitution R101D or R101E and the amino acid substitutions S3T, V4I, and V205I according to the numbering of SEQ ID NO: 2. Even more preferably, the protease has at least 90%, at least 95%, at least 96%, at least 97%, at least 98%, at least 99%, or 100% sequence identity to SEQ ID NO: 1 and comprises compared to SEQ ID NO: 1 the amino acid substitution R101D or R101E (preferably R101E) and the amino acid substitutions S3T, V4I, and V205I according to the numbering of SEQ ID NO: 2. Preferably, the protease has 100% sequence identity to SEQ ID NO: 1 and comprises compared to SEQ ID NO: 1 the amino acid substitution R101D or R101E (preferably R101E) and the amino acid substitutions S3T, V4I, and V205I according to the numbering of SEQ ID NO: 2, i.e., the protease comprises compared to SEQ ID NO: 1 only four amino acid exchanges.

Preferably, the protease has at least 80% sequence identity to SEQ ID NO: 1 and comprises compared to SEQ ID NO: 1 at least two additional negative charges in the loop region of residues 98 to 104 according to the numbering of SEQ ID NO: 2, wherein compared to SEQ ID NO: 1 the protease comprises one or more conservative amino acid exchanges as described herein.

Preferably, compared to SEQ ID NO: 1 the protease comprises at least 1, at least 2, at least 3, at least 4, at least 5, at least 6, at least 10, at least 15, at least 20, at least 30 or at least 40 conservative amino acid exchanges.

Preferably, compared to SEQ ID NO: 1 a protease described herein can comprise 1, 2, 3, 4, 5, 6, 7, 8, 9, or 10 conservative amino acid exchanges in addition to the modifications resulting in at least two additional negative charges in the loop region of residues 98 to 104, preferably in addition to the substitution R101D or R101E and preferably the amino acid substitutions S3T, V4I, and V205I according to the numbering of SEQ ID NO: 2.

Preferably, the protease has at least 80% sequence identity to SEQ ID NO: 1 and comprises compared to SEQ ID NO: 1 at least two additional negative charges in the loop region of residues 98 to 104 according to the numbering of SEQ ID NO: 2, wherein compared to SEQ ID NO: 1 the remaining difference in amino acid sequence is due to conservative amino acid exchanges as described herein.

In a preferred embodiment, the protease comprises compared to SEQ ID NO: 1 one or more substitutions at positions according the numbering of SEQ ID NO: 2 selected from the group consisting of 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and 274.

Preferably the variant protease comprises an amino acid sequence which is at least 80% identical to SEQ ID NO: 1 and wherein the amino acid sequence of the protease comprises compared to SEQ ID NO: 1 at least two additional negative charges in the loop region of residues 98 to 104 according to the numbering of SEQ ID NO: 2 and protease comprises compared to SEQ ID NO: 1 one or more substitutions at positions according the numbering of SEQ ID NO: 2 selected from the group consisting of 3, 4, 9, 15, 24, 27, 33, 36, 45, 55, 57, 58, 59, 61, 68, 76, 77, 87, 95, 96, 97, 98, 99, 100, 101, 102, 103, 104, 106, 117, 118, 120, 123, 124, 128, 129, 130, 131, 136, 137, 143, 154, 156, 160, 161, 163, 167, 170, 171, 172, 185, 194, 195, 199,205, 206, 209, 217, 218, 222, 224, 232, 235, 236, 238, 244, 245, 248, 252, 261, 262, and 274.

Preferably, the protease has at least 80% sequence identity to SEQ ID NO: 1 as described herein and comprises compared to SEQ ID NO: 1 at least two, three, or four additional negative charges, more preferably three additional negative charges, most preferably two additional negative charges in the loop region of residues 98 to 104 according to the numbering of SEQ ID NO: 2 compared to the region of SEQ ID NO: 1 corresponding to residues 98 to 104 of SEQ ID NO: 2.

Preferably, the at least two additional negative charges in the loop region of residues 98 to 104 according to the numbering of SEQ ID NO: 2 are obtained by one or more amino acid alterations selected from the group consisting of substitutions, deletions and insertions, preferably by substitutions. Preferably, the at least two additional negative charges in the loop region of residues 98 to 104 according to the numbering of SEQ ID NO: 2 are obtained by one or more amino acid alterations selected from the group consisting D99E, R101D and R101E.

Preferably, in the protease the at least two additional negative charges compared to SEQ ID NO: 1 in the loop region of residues 98 to 104 are caused by one or more amino acid substitutions at amino acid position according the numbering of SEQ ID NO: 2 selected from the group consisting of 98, 99, 100, 101, 102, 103, and 104, preferably at position 101.

In a preferred embodiment, the protease comprises an amino acid sequence which comprises compared to SEQ ID NO: 1 the amino acid substitution R101E or R101D according to the numbering of SEQ ID NO: 2. In another preferred embodiment, the at least two additional negative charges compared to SEQ ID NO: 1 in the loop region of residues 98 to 104 are not caused by the amino acid substitution R101E or R101D.

In a preferred embodiment, the loop sequence 98-104 has compared to SEQ ID NO: 1 two additional negative charges with the following sequence ADGEGAI, ADGDGAI, ADGDGSV, ADGEGSV, AADGEGSV, or ASEGEGSV with longer sequences having an insertion in the loop sequence.

In another embodiment of the present invention, the protease comprising as described herein an amino acid sequence which is at least 80% identical to SEQ ID NO: 1 and wherein the amino acid sequence of the protease comprises compared to SEQ ID NO: 1 at least two additional negative charges in the loop region of residues 98 to 104 according to the numbering of SEQ ID NO: 2, further comprises according to the numbering of SEQ ID NO: 2 at least one of the amino acid residues selected from the group consisting of
a. threonine or serine at position 3 (3T or 3S),
b. isoleucine or valine at position 4 (41 or 4V),
c. serine, alanine, threonine or arginine at position 63 (63S, 63A, 63T or 63R),
d. threonine, aspartic acid or glutamic acid at position 156 (156T, 156D, or 156E),
e. serine or proline at position 194 (194S or 194P),
f. serine, valine, or methionine at position 199 (199S, 199V, or 199M)
g. isoleucine or valine at position 205 (205I or 205V); and
h. aspartic acid, glutamic acid, glutamine, glycine at position or leucine at position 217 (217D, 217E, 217Q, 217G or 217L).

In a preferred embodiment, the protease comprises an amino acid sequence which is at least 80% identical to SEQ ID NO: 1 and which comprises compared to SEQ ID NO: 1 the amino acid substitution R101E or R101D according to the numbering of SEQ ID NO: 2 and wherein the protease according to the numbering of SEQ ID NO: 2 comprises at least one of the amino acid residues selected from the group consisting of
a. threonine or serine at position 3 (3T or 3S),
b. isoleucine or valine at position 4 (41 or 4V),
c. serine, alanine, threonine or arginine at position 63 (63S, 63A, 63T or 63R),
d. threonine, aspartic acid or glutamic acid at position 156 (156T, 156D, or 156E),
e. serine or proline at position 194 (194S or 194P),
f. serine, valine, or methionine at position 199 (199S, 199V, or 199M)
g. isoleucine or valine at position 205 (205I or 205V); and
h. aspartic acid, glutamic acid, glutamine, glycine at position or leucine at position 217 (217D, 217E, 217Q, 217G or 217L).

Preferably, the protease described herein comprises compared to SEQ ID NO: 1 the amino acid substitution R101E or R101D and the amino acid substitutions S3T, V4I, and V205I according to the numbering of SEQ ID NO: 2.

In a another embodiment, the protease comprises an amino acid sequence which is at least 80% identical to SEQ ID NO: 1 and the protease comprises compared to SEQ ID NO: 1 the amino acid substitution R101E or R101D and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T,D and L262N,Q,D according to the numbering of SEQ ID NO: 2.

In a another embodiment, the protease comprises an amino acid sequence which is at least 80% identical to SEQ ID NO: 1 and the protease comprises compared to SEQ ID NO: 1 the amino acid substitutions R101E and S156D and/or L262E, and optionally at least one further mutation selected from 1104T, H120D, Q137H, S141H, R145H and S163G according to the numbering of SEQ ID NO: 2.

Preferably, the protease has an additional mutation at position 217 according to the numbering of SEQ ID NO: 2, preferably L217Q, L217D, L217E, or L217G.

In a preferred embodiment, the protease comprises an amino acid sequence selected from the group consisting of
amino acid sequence of SEQ ID NO: 3,
amino acid sequence of SEQ ID NO: 3, wherein the amino acid sequence comprises at least one additional amino acid substitution selected from the group consisting of
threonine at position 3 (3T);
isoleucine at position 4 (41);
serine, alanine, threonine or arginine at position 63 (63S, 63A, 63T, or 63R);
threonine, aspartic acid or glutamic acid at position 156 (156T, 156D, or 156E); serine or proline at position 194 (194S or 194P);
methionine or serine at position 199 (199M or 199S);
isoleucine at position 205 (205I); and
aspartic acid, glutamic acid, glutamine or glycine at position 217 (217D, 217E, 217Q or 217G);
amino acid sequence of SEQ ID NO: 4, and
amino acid sequence of SEQ ID NO: 4, wherein the amino acid sequence comprises at least one additional amino acid substitution selected from the group consisting of
serine at position 3 (3S);
valine at position 4 (4V);
serine, alanine, threonine or arginine at position 63 (63S, 63A, 63T, or 63R);
threonine, aspartic acid or glutamic acid at position 156 (156T, 156D, or 156E); serine or proline at position 194 (194S or 194P);
methionine or serine at position 199 (199M or 199S);
valine at position 205 (205V); and aspartic acid, glutamic acid, glutamine or glycine at position 217 (217D, 217E, 217Q or 217G).

Preferably, the amino acid sequence of the protease comprises compared to SEQ ID NO: 1 alanine at position 103 (103A) and isoleucine at position 104 (1041) according to the numbering of SEQ ID NO: 2, more preferably, 101R, 1041, and 103A.

In a further preferred embodiment, the amino acid sequence of the protease compared to SEQ ID NO: 1 does not comprises an additional amino acid residue in the loop region from position 98 to 104 according to the numbering of SEQ ID NO: 2. Preferably, the amino acid sequence of the protease compared to SEQ ID NO: 1 does not comprises an additional amino acid residue between positions 42-43, 51-55, 155-165, 187-189, 217-218, or 218-219 according to the numbering of SEQ ID NO: 2.

Proteases, including serine proteases, according to the invention have “proteolytic activity” (also referred to as “protease activity”). This property is related to hydrolytic activity of a protease (proteolysis, which means hydrolysis of peptide bonds linking amino acids together in a polypeptide chain) on protein containing substrates, e.g. casein, haemoglobin and BSA. Quantitatively, proteolytic activity is related to the rate of degradation of protein by a protease or proteolytic enzyme in a defined course of time. The methods for analyzing proteolytic activity are well-known in the literature (see e.g. Gupta et al. (2002), Appl. Microbiol. Biotechnol. 60: 381-395).

For instance, proteolytic activity and thereby the effect of an inhibitor on the proteolytic activity as such can be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of yellow color of free pNA which can be quantified by measuring OD405.

To determine changes in proteolytic activity over time, the “initial enzymatic activity” of a protease is measured under defined conditions at time zero and at a certain point in time later. By dividing the latter activity with the activity at time point zero the residual activity can be calculated (x %). The x % value measured shall preferably equal the 100%-value indicating no loss in activity. By comparison of the 100%-value with the x %-value, a potential loss of proteolytic activity can be determined in its extent.

### Protease Inhibitor

The protease inhibitor in the composition of the present invention is a peptide aldehyde or a derivative thereof, preferably the protease inhibitor in the composition of the present invention is a peptide aldehyde, a peptide aldehyde hydrosulfite adduct, or a combination thereof.

The protease inhibitor may comprise 2, 3, 4, 5 or 6 amino acid residues. The N-terminus of the peptide aldehyde may be H or may be protected by an N-terminal protection group (herein referred to as Z).

The protease inhibitor may have an inhibition constant to a serine protease Ki (M, mol/L) of 1 E12-1 E-03; more preferred 1 E-11-1 E-04; even more preferred: 1 E-10-1 E-05; even more preferred 1E-10-1 E-06; most preferred 1 E-09-1 E-07.

Preferably, the protease inhibitor is a peptide aldehyde.

Preferably, the peptide aldehyde has the general formula (I) A4-A3-A2-A1, where
A1 is an aldehyde (CHO) or aldehyde analogue (COCX3, COCHX2, COCH2X or COCH3, where X is a halogen atom) with a possibility to bind to the serine in the active site of the serine hydrolase.
A2 is an amino acid residue with aromatic or aliphatic side chain with its C-terminal carbon included in A1 described above. Preferred amino acids are D- or L-Tyr (p-tyrosine), m-tyrosine, 3,4-dihydroxy-phenylalanine, Leu, Phe, Val, Met, Nva, Nle;
A3 if present may a non-bulky linker, preferably hydrophobic, consisting of either 1-3 small amino acids (e.g. Gly, Ala, Leu, norleucine, norvaline);
A4 if present may be a protection group (e.g. an N-protection group as described below), or any amino acid, preferably Gly or Val

The peptide aldehyde may have the formula (II) B2-B1-B0-R wherein:
a) R is hydrogen, CH3, CX3, CHX2, or CH2X, wherein X is a halogen atom;
b) B0 is a single amino acid residue;
c) B1 is 1-2 amino acid residues; and
d) B2 consists of one or more amino acid residues (preferably one or two), optionally comprising an N-terminal protection group or an N-terminal protection group attached to B1.

In the above formula (II), B0 may be an L or D-amino acid with an optionally substituted aliphatic or aromatic side chain, preferably D- or L-Tyr (p-tyrosine), m-tyrosine, 3,4-dihydroxyphenylalanine, Leu, Phe, Val, Gly, Met, Trp, Ile, Nva or Nle.

B1 may be 1-2 amino acid residues, preferably 2, with a small optionally substituted aliphatic side chain, preferably selected from Ala, Cys, Gly, Leu, Arg, Pro, Ser, Ile, Thr, Val, Nva, or Nle, most preferred selected from Ala, Gly, Leu, Nva or Nie. The most preferred combinations are, Gly-Ala, Ala-Ala, Gly-Gly, and Gly-Leu.

B2 of formula (II) ay be an N-capping group known from protein chemistry, preferably selected from benzyloxycarbonyl (Cbz), p-methoxybenzyl carbonyl (MOZ), benzyl (Bn), benzoyl (Bz), pmethoxybenzyl (PMB), p-methoxyphenyl (PMP), formyl, acetyl, methyloxy, methyloxycarbonyl/methyl carbamate, or methyl urea, or B2 may be any 1-2 amino acid residues without specifying structure, preferably Gly or Val, optionally comprising a protection group as described above.

More particularly, the peptide aldehyde may be Z-GGY-H, Z-GGF-H, Z-GGG-H, ZGAG-H, ZRAY—H, Ac-GAY-H, Z-GAY-H, Z-GAL-H, Z-GAF-H, Z-GAV-H, Z-RVY-H, Z-LVY-H, Z-VAL-H, AcLGAY—H, Ac-FGAY-H, Ac-YGAY-H, Ac-FGVY-H, Ac-FGAM-H, Ac-WLVY-H, MeO-CO-VALH, MeO-CO-LLY-H, MeOCO-FGAL-H, MeO-FGAF-H, MeNCO-FGAL-H, Z-VAL-CF3, wherein Z is preferably benzyloxycarbonyl, Me is methyl and Ac is acetyl.

Alternatively, the peptide aldehyde may have the formula (III) B₂-B₁-B0-R wherein:
R is hydrogen, CH3, CX3, CHX2, or CH2X, wherein X is a halogen atom;
B0 is a single amino acid residue;
B1 is a single amino acid residue; and
B2 consists of one or more amino acid residues (preferably one or two), optionally comprising an N-terminal protection group.

In the above formula (III), B0 may be an L or D-amino acid with an optionally substituted aliphatic or aromatic side chain, preferably D- or L-Tyr (p-tyrosine), m-tyrosine, 3,4-dihydroxyphenylalanine, Leu, Phe, Val, Met, Nva or Nle.

B1 of formula (III) may be a residue with a small optionally substituted aliphatic side chain, preferably Ala, Cys, Gly, Pro, Ser, Thr, Val, Nva, or Nle.

B2 of formula (III) may be either one residue B2 with either a small aliphatic side chain (preferably, Gly, Ala, Thr, Val or Leu) or Arg or Gln; optionally comprising a N-terminal protection group, selected from the “aromate” or “small” protection groups described below; or B2 may be two residues B3-B2′ where B2′ is like B2 above and B3 is a residue with an hydrophobic or aromatic side chain (preferably Phe, Tyr, Trp, m-tyrosine, 3,4-dihydroxyphenylalanine, phenylglycine, Leu, Val, Nva, Nle or Ile) optionally comprising a N-protection group.

Preferably B₂ of formula (III) allows for placing an aromatic or hydrophobic system in the “fourth position” counting from the aldehyde, this could be N-“aromate”-B₂, where B₂ is like described above and “aromate” protection group contain an aromatic or hydrophobic group such as benzyloxycarbonyl (Cbz), p-methoxybenzyl carbonyl (MOZ), benzyl (Bn), benzoyl (Bz), p-methoxybenzyl (PMB) or p-methoxyphenyl (PMP). Alternatively, most preferred, B2 may be a dipeptide of the form N-“small”-B3-B2′, where B2′ and B3 are like described above with a “small” N-terminal protection group attached such as formyl, acetyl, methyloxy, or methyloxycarbonyl.

In a particularly preferred embodiment, the peptide aldehyde is a tripeptide aldehyde, preferably selected from a compound of formula (IV) or a salt thereof:

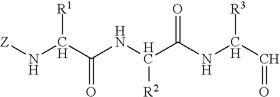

R¹, R², R³ and Z within formula (IV) are defined as follows:
R¹ is a group such that NH—CHR¹—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle. Preferably, R¹ is a group such that NH—CHR¹—CO is an L or D-amino acid residue of Ala, Val, Gly, Arg, Leu, Phe, Ile, His or Thr. More preferably, R¹ is a group such that NH—CHR¹—CO is an L or D-amino acid residue of Ala, Val, Gly, Arg, Leu, Ile or His.
R² is a group such that NH—CHR²—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle. Preferably, R² is a group such that NH—CHR²—CO is an L or D-amino acid residue of Ala, Cys, Gly, Pro, Ser, Thr, Val, Nva or Nle. More preferably, R² is a group such that NH—CHR²—CO is an L or D-amino acid residue of Ala, Gly, Pro or Val.
R³ is a group such that NH—CHR³—CO is an L or D-amino acid residue of Tyr, m-tyrosine, 3,4-dihydroxyphenylalanine, Phe, Val, Ala, Met, Nva, Leu, Ile or Nle or other non-natural amino acids carrying alkyl groups. Preferably, R³ is a group such that NH—CHR³—CO is an L or D-amino acid residue of Tyr, Phe, Val, Ala or Leu.

In one embodiment, R¹ and R² of formula (IV) are groups such that NH—CHR¹—CO and/or NH—CHR²—CO are non-polar amino acids, preferably independently from each other selected from an L or D-amino acid residue of Ala, Val, Gly and Leu. R³ is a group such that NH—CHR³—CO is an L or D-amino acid residue of Tyr, Phe, Val, Ala or Leu.

In one embodiment, R¹ of formula (IV) is a group such that NH—CHR¹—CO is an L or D-amino acid residue of Gly or Val, R² is a group such that NH—CHR²—CO is an L or D-amino acid residue of Ala, and R³ is a group such that NH—CHR³—CO is an L or D-amino acid residue of Tyr, Ala, or Leu.

In one embodiment, at least two selected from R¹, R² and R³ of formula (IV) are groups such that NH—CHR¹—CO and/or NH—CHR²—CO and/or NH—CHR³—CO are non-polar amino acid residues, preferably independently from each other selected from an L or D-amino acid residue of Ala, Val, Gly and Leu.

In one embodiment, R¹ of formula (IV) is a group such that NH—CHR¹—CO is an L or D-amino acid residue of Val, R² is a group such that NH—CHR²—CO is an L or D-amino acid residue of Ala, and R³ is a group such that NH—CHR³—CO is an L or D-amino acid residue of Leu.

Z of formula (IV) is selected from hydrogen, an N-terminal protection group, and one or more amino acid residues optionally comprising an N-terminal protection group. Preferably, Z is an N-terminal protection group.

In a preferred embodiment, the liquid compositions of the invention comprise at least one peptide aldehyde (component (a)) selected from compounds according to formula (IV), wherein
R¹ is a group such that NH—CHR¹—CO is an L or D-amino acid residue of Gly or Val, R² is a group such that NH—CHR²—CO is an L or D-amino acid residue of Ala, and R³ is a group such that NH—CHR³—CO is an L or D-amino acid residue of Tyr, Ala, or Leu.

and
the N-terminal protection group Z, preferably, Z is selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), or tert-butyloxycarbonyl (Boc),

and wherein the at least one peptide aldehyde is comprised in amounts in the range of about 0.05% to 0.8% by weight relative to the total weight of the liquid composition, wherein the amount relates to 100% active content. Preferably, the peptide aldehyde is comprised in amounts in the range of about 0.1% to 0.6% by weight, of about 0.12% to 0.5% by weight, of about 0.15% to 0.4%, or of about 0.2% to 0.35% by weight, all relative to the total weight of the liquid composition.

In a more preferred embodiment, at least one peptide aldehyde is selected from compounds according to formula (IV), wherein
R¹ is a group such that NH—CHR¹—CO is an L or D-amino acid residue of Val, R² is a group such that NH—CHR²—CO is an L or D-amino acid residue of Ala, and R³ is a group such that NH—CHR³—CO is an L or D-amino acid residue of Leu.

Even more preferably, the N-terminal protection group Z of the peptide aldehyde is benzyloxycarbonyl (Cbz).

In one embodiment, R¹ and R² of formula (IV) is a group such that NH—CHR¹—CO and NH—CHR²—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle, preferably independently from each other selected from an L or D-amino acid residue of Ala, Val, Gly and Leu;

In one embodiment, R³ of formula (IV) is a group such that NH—CHR³—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle, or wherein R³ is (CH₃)₃SiCH₂, preferably independently from each other selected from an L or D-amino acid residue of Tyr, Phe, Val, Ala and Leu.

In one embodiment, R¹ of formula (IV) is a group such that NH—CHR¹—CO is an L or D-amino acid residue of Ala, Val, Gly, Arg, Leu, Phe or Thr.

In one embodiment, R² of formula (IV) is a group such that NH—CHR²—CO is an L or D-amino acid residue of Ala, Cys, Gly, Pro, Ser, Thr, Val, Nva or Nle.

In one embodiment, R³ of formula (IV) is a group such that NH—CHR³—CO is an L or D-amino acid residue of Tyr, m-tyrosine, 3,4-dihydroxyphenylalanine, Phe, Val, Ala, Met, Nva, Leu, Ile or Nle.

The peptide aldehydes described herein may be prepared from the corresponding amino acid whereby the C-terminal end of said amino acid is converted from a carboxylic group to an aldehyde group. Such aldehydes may be prepared by known processes, for instance as described in U.S. Pat. No. 5,015,627, EP185930, EP583534, and DE3200812.

The protease inhibitor may also be a hydrosulfite adduct of a peptide aldehyde. The peptide aldehyde hydrosulfite adduct may be of the formula (V) X—Br—NH—CHR—CHOH—SO3M, wherein:
a) M is H (hydrogen) or an alkali metal;
b) R is a group such that NH—CHR—CO is an L or D-amino acid residue;
c) B₁ is one amino acid residue; and
d) X consists of one or more amino acid residues, optionally comprising an N-terminal protection group as described herein.

Preferably, R of formula (V) is a group such that NH—CHR—CO is an L or D-amino acid residue of Tyr, m-tyrosine, 3,4-dihydroxyphenylalanine, Phe, Val, Met, Nva, Leu, Ile or Nie.

Preferably, B1 of formula (V) is a residue of Ala, Cys, Gly, Pro, Ser, Thr, Val, Nva, or Nle.

Preferably, X of formula (V) is B2, B3-B2, Z-B2, or Z-B3-B2, wherein B2 and B3 each represents one amino acid residue, and Z is an N-terminal protection group.

Preferably, B2 of formula (V) is a residue of Val, Gly, Ala, Arg, Leu, Phe or Thr.

Preferably, B3 of formula (V) is a residue of Phe, Tyr, Trp, Phenylglycine, Leu, Val, Nva, Nle or Ile.

Most preferably, the protease inhibitor is a peptide aldehyde, preferably Z-GAY-H or Z-VAL-H, particularly preferred Z-VAL-H.

The peptide aldehyde or peptide aldehyde hydrosulfite adduct preferably comprises an N-terminal protection group. The N-terminal protection group may be selected from formyl, acetyl (Ac), benzoyl (Bz), trifluoroacetyl, fluorenylmethyloxycarbonyl (Fmoc), methoxysuccinyl, aromatic and aliphatic urethane protecting groups, benzyloxycarbonyl (Cbz), tert-butyloxycarbonyl (Boc), adamantyloxycarbonyl, p-methoxybenzyl carbonyl (MOZ), benzyl (Bn), p-methoxybenzyl (PMB) or p-methoxyphenyl (PMP), methoxycarbonyl (Moc); methoxyacetyl (Mac); methyl carbamate, a methylamino carbonyl/methyl urea group, tityl (Trt), 3,5-dimethoxyphenylisoproxycarbonyl (Ddz), 2-(4-biphenyl)isopropoxycarbonyl (Bpoc), 2-nitrophenylsulfenyl (Nps), 2-(4-nitrophenylsulfonyl)ethoxycarbonyl (Nsc), 1,1-dioxobenzo[b]thiophene-2-ylmethyloxycarbonyl (Bsmoc), (1,1-dioxonaphtho[1,2-b]thiophene-2-yl)methyloxycarbonyl (α-Nsmoc), 1-(4,4-dimethyl-2,6-dioxocyclohex-1-ylidene)-3-methylbutyl (ivDde), 2,7-di-tert-butyl-Fmoc (Fmoc*), 2-fluoro-Fmoc (Fmoc(2F)), 2-monoisooctyl-Fmoc (mio-Fmoc) and 2,7-diisooctyl-Fmoc (dio-Fmoc), tetrachlorophthaloyl (TCP), 2-phenyl(methyl)sulfonio)ethyloxycarbonyl tetrafluoroborate (Pms), ethanesulfonylethoxycarbonyl (Esc), 2-(4-sulfophenylsulfonyl)ethoxycarbonyl (Sps), allyloxycarbonyl (Alloc), o-nitrobenzenesulfonyl (oNBS), 2,4-dinitrobenzenesulfonyl (dNBS), Benzothiazole-2-sulfonyl (Bts), 2,2,2-trichloroethyloxycarbonyl (Troc), dithiasuccinoyl (Dts), p-nitrobenzyloxycarbonyl (pNZ), α-Azidoacids, Propargyloxycarbonyl (Poc), o-Nitrobenzyloxycarbonyl (oNZ), 4-Nitroveratryloxycarbonyl (NVOC), 2-(2-Nitrophenyl)propyloxycarbonyl (NPPOC), 2-(3,4-Methylenedioxy-6-nitrophenyl)propyloxycarbonyl (MNPPOC), 9-(4-Bromophenyl)-9-fluorenyl (BrPhF), Azidomethyloxycarbonyl (Azoc), Hexafluoroacetone (HFA), 2-Chlorobenzyloxycarbonyl (CI-Z), Trifluoroacetyl (tfa), 2-(Methylsulfonyl)ethoxycarbonyl (Msc), Tetrachlorophthaloyl (TCP), Phenyldisulphanylethyloxycarbonyl (Phdec), 2-Pyridyldisulphanylethyloxycarbonyl (Pydec), or 4-Methyltrityl (Mtt).

If Z in the formula above is one or more amino acid residue(s) comprising an N-terminal protection group, the N-terminal protection group is preferably a small aliphatic group, e.g., formyl, acetyl, fluorenylmethyloxycarbonyl (Fmoc), tert-butyloxycarbonyl (Boc), methoxycarbonyl (Moc); methoxyacetyl (Mac); methyl carbamate or a methylamino carbonyl/methyl urea group. In the case of a tripeptide, the N-terminal protection group is preferably a bulky aromatic group such as benzoyl (Bz), benzyloxycarbonyl (Cbz), p-methoxybenzyl carbonyl (MOZ), benzyl (Bn), pmethoxybenzyl (PMB) or p-methoxyphenyl (PMP).

Further suitable N-terminal protection groups are described in Greene's Protective Groups in Organic Synthesis, Fifth Edition by Peter G. M. Wuts, published in 2014 by John Wiley & Sons, Inc and in Isidro-Llobet et al., Amino Acid-Protecting Groups, Chem. Rev. 2009 109(6), 2455-2504.

Preferably, the N-terminal protection group is selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), or tert-butyloxycarbonyl (Boc).

Preferred peptide aldehyde protease inhibitors are selected from the group consisting of: More particularly, the peptide aldehyde may be Z-GGY-H, Z-GGF-H, Z-GGG-H, ZGAG-H, ZRAY-H, Ac-GAY-H, Z-GAY-H, Z-GAL-H, Z-VAL-H, Z-VAL-CF3, Z-GAF-H, Z-GAF-CF3, Z-GAVH, Z-GGY-H, Z-GGF-H, Z-RVY-H, Z-LVY-H, Ac-LGAY-H, Ac-FGAY-H, Ac-YGAY-H, Ac-FGALH, Ac-FGAF-H, Ac-FGVY-H, Ac-FGAM-H, AcWLVY-H, MeO-CO-VAL-H, MeNCO-VAL-H, MeO-CO-FGAL-H, MeO-CO-FGAF-H, MeSO2-FGAL-H, MeSO2VAL-H, PhCH2O(OH)(O)P-VAL-H, EtSO2-FGAL-H, PhCH2SO2VAL-H, PhCH2O(OH)(O)P-LAL-H, PhCH2O(OH)(O)P-FAL-H, and MeO(OH)(O)P-LGAL-H, wherein Me is methyl, Ac is acetyl and Z is preferably benzyloxycarbonyl. The most preferred peptide aldehyde is Z-GAY-H or Z-VAL-H, further preferred Z-VAL-H.

The peptide aldehyde hydrosulfite adduct may be selected from the group consisting of CbzRA-NHCH(CH2C5H4OH)C(OH)(SO3M)-H, Ac-GANHCH(CH2C5H4OH)C(OH)(SO3M)-H, CbzGA-NHCH(CH2C5H4OH)C(OH)(SO3M)-H, Cbz-GANHCH(CH2CH(CH3)2))C(OH)(SO3M)-H, Cbz-VA-NHCH(CH2CH(CH3)2))C(OH)(SO3M)-H, Cbz-GA-NHCH(CH2Ph)C(OH)(SO3M)-H, Cbz-GA-NHCH(CH(CH3)2)C(OH)(SO3M)-H, Cbz-GGNHCH(CH2C5H4OH)C(OH)(SO3M)-H, Cbz-GG-NHCH(CH2Ph)C(OH)(SO3M)-H, Cbz-RVNHCH(CH2C5H4OH)C(OH)(SO3M)-H, CbzLV-NHCH(CH2C5H4OH)C(OH)(SO3M)-H, Ac-LGANHCH(CH2C5H4OH)C(OH)(SO3M)-H, AcFGA-NHCH(CH2C5H4OH)C(OH)(SO3M)-H, Ac-YGA-NHCH(CH2C5H4OH)C(OH)(SO3M)-H, Ac-FGA-NHCH(CH2CH(CH3)2))C(OH)(SO3M)-H, AcFGA-NHCH(CH2Ph)C(OH)(SO3M)-H, AcFGV-NHCH(CH2C5H4OH)C(OH)(SO3M)-H, Ac-FGANHCH(CH2CH2SCH3)(SO3M)-H, AcWLV-NHCH(CH2C5H4OH)C(OH)(SO3M)-H, MeO-COVANHCH(CH2CH(CH3)2))C(OH)(SO3M)-H, MeNCO-VANHCH(CH2CH(CH3)2))C(OH)(SO3M)-H, MeO-CO-FGANHCH(CH2CH(CH3)2))C(OH)(SO3M)-H, MeO-CO-FGA-NHCH(CH2Ph)-C(OH)(SO3M)-H, MeSOrFGA-NHCH(CH2CH(CH3)2))C(OH)(SO3M)-H, MeSOrVANHCH(CH2CH(CH3)2))C(OH)(SO3M)-H, PhCH2O(OH)(O)P-VANHCH(CH2CH(CH3)2))C(OH)(SO3M)-H, EtSOrFGA-NHCH(CH2CH(CH3)2))C(OH)(SO3M)-H, PhCH2SOrVANHCH(CH2CH(CH3)2))C(OH)(SO3M)-H, PhCH2O(OH)(O)P-LANHCH(CH2CH(CH3)2))C(OH)(SO3M)-H, PhCH2O(OH)(O)P-FANHCH(CH2CH(CH3)2))C(OH)(SO3M)-H, MeO(OH)(O)P-LGANHCH(CH2CH(CH3)2))C(OH)(SO3M)-H, and F-urea-RVNHCH(CH2C6H4OH)C(OH)(SO3M)-H where M=negative charge, H, Na, or K or another counterion. The most preferred peptide aldehyde hydrosulfite adduct is the hydrosulfite adduct of Z-GAY-H or Z-VAL-H

### Secondary Enzymes

The composition of the present invention further comprises at least one second enzyme different from the protease described above. Preferably, the at least one second enzyme is a detergent enzyme In one embodiment, the second enzyme is classified as an oxidoreductase (EC 1), a transferase (EC 2), a hydrolase (EC 3), a lyase (EC 4), an isomerase (EC 5), or a Ligase (EC 6) (EC-numbering according to Enzyme Nomenclature, Recommendations (1992) of the Nomenclature Committee of the International Union of Biochemistry and Molecular Biology including its supplements published 1993-1999).

In a preferred embodiment, the second enzyme is a hydrolase (EC 3), in one embodiment a glycosidase (EC 3.2) or a peptidase (EC 3.4). In one embodiment, enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC3.1.1.3), a phytase (EC 3.1.3.8), a nuclease (EC 3.1.11 to EC 3.1.31), and a protease. In more preferred embodiment, the second enzyme is selected from the group consisting of oxidoreductase, transferase, hydrolase, lyase, isomerase, ligase, aminopeptidase, amylase, asparaginase, carbohydrase, carboxypeptidase, catalase, cellulase, chitinase, cutinase, cyclodextrin glycosyltransferase, deoxyribonuclease, esterase, alpha-galactosidase, betagalactosidase, glucoamylase, alpha-glucosidase, beta-glucosidase, hyaluronic acid synthase, invertase, laccase, lipase, mannosidase, mutanase, oxidase, a pectinolytic enzyme, peroxidase, phytase, polyphenoloxidase, protease, ribonuclease, transglutaminase, dispersin, and orxylanase. In particular, the second enzyme is selected from the group consisting of amylase, cellulase, mannanase, lipase, dispersin, and DNase. Preferably, the second enzyme is selected from the group consisting of amylase, cellulase, mannanase, and lipase, preferably, a lipase.

### Lipases

At least one enzyme may be selected from the group of lipases. “Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases include those of bacterial or fungal origin.

The methods for determining lipolytic activity are well-known in the literature (see e.g. Gupta et al. (2003), Biotechnol. Appl. Biochem. 37, p. 63-71). E.g. the lipase activity may be measured by ester bond hydrolysis in the substrate para-nitrophenyl palmitate (pNP-Palmitate, C:16) and releases pNP which is yellow and can be detected at 405 nm.

“Lipolytic activity” means the catalytic effect exerted by a lipase, which may be provided in lipolytic units (LU). For example, 1 LU may correspond to the amount of lipase which produces 1 μmol of titratable fatty acid per minute in a pH stat. under the following conditions: temperature 30° C.; pH=9.0; substrate may be an emulsion of 3.3 wt. % of olive oil and 3.3% gum arabic, in the presence of 13 mmol/l Ca²⁺ and 20 mmol/l NaCl in 5 mmol/l Tris-buffer.

In one aspect of the invention, a suitable lipase (component (b)) is selected from the following: lipases from Humicola (synonym Thermomyces), e.g. from H. lanuginosa (T. lanuginosus) as described in EP 258068, EP 305216, WO 92/05249 and WO 2009/109500 or from H. insolens as described in WO 96/13580; lipases derived from Rhizomucor miehei as described in WO 92/05249; lipase from strains of Pseudomonas (some of these now renamed to Burkholderia), e.g. from P. alcaligenes or P. pseudoalcaligenes (EP 218272, WO 94/25578, WO 95/30744, WO 95/35381, WO 96/00292), P. cepacia (EP 331376), P. stutzeri (GB 1372034), P. fluorescens, Pseudomonas sp. strain SD705 (WO 95/06720 and WO 96/27002), P. wisconsinensis (WO 96/12012), Pseudomonas mendocina (WO 95/14783), P. glumae (WO 95/35381, WO 96/00292); lipase from Streptomyces griseus (WO 2011/150157) and S. pristinaespiralis (WO 2012/137147), GDSL-type Streptomyces lipases (WO 2010/065455); lipase from Thermobifida fusca as disclosed in WO 2011/084412; lipase from Geobacillus stearothermophilus as disclosed in WO 2011/084417; Bacillus lipases, e.g. as disclosed in WO 00/60063, lipases from B. subtilis as disclosed in Dartois et al. (1992), Biochemica et Biophysica Acta, 1131, 253-360 or WO 2011/084599, B. stearothermophilus (JP S64-074992) or B. pumilus (WO 91/16422); lipase from Candida antarctica as disclosed in WO 94/01541; cutinase from Pseudomonas mendocina (U.S. Pat. No. 5,389,536, WO 88/09367); cutinase from Magnaporthe grisea (WO 2010/107560); cutinase from Fusarum solani pisi as disclosed in WO 90/09446, WO 00/34450 and WO 01/92502; and cutinase from Humicola lanuginosa as disclosed in WO 00/34450 and WO 01/92502.

Such suitable lipase variants are e.g. those which are developed by methods as disclosed in WO 95/22615, WO 97/04079, WO 97/07202, WO 00/60063, WO 2007/087508, EP 407225 and EP 260105.

Suitable lipases include also those, which are variants of the above described lipases which have lipolytic activity. In one embodiment lipase variants include variants with at least 40 to 100% identity when compared to the full length polypeptide sequence of the parent enzyme as disclosed above. In one embodiment lipase variants having lipolytic activity are at least 70%, at least 75%, at least 80%, at least 85%, at least 90%, at least 91%, at least 92%, at least 93%, at least 94%, at least 95%, at least 96%, at least 97%, at least 98% or at least 99% identical to the full length polypeptide sequence of the parent enzyme as disclosed above.

In another embodiment, the invention relates to lipase variants comprising conservative mutations not pertaining the functional domain of the respective lipase. Lipase variants of this embodiment having lipolytic activity may be at least 70%, at least 75%, at least 80%, at least 85%, at least 90%, at least 91%, at least 92%, at least 93%, at least 94%, at least 95%, at least 96%, at least 97%, at least 98% or at least 99% similar to the full length polypeptide sequence of the parent enzyme.

In one embodiment, lipase variants have lipolytic activity according to the present invention when said lipase variants exhibit increased lipolytic activity when compared to the parent lipase.

Commercially available lipase enzymes include but are not limited to those sold under the trade names Lipolase™, Lipex™, Lipolex™ and Lipoclean™ (Novozymes A/S), Lumafast (originally from Genencor), Preferenz L (DuPont), and Lipomax (Gist-Brocades/now DSM).

In one embodiment, lipase is selected from fungal triacylglycerol lipase (EC class 3.1.1.3). Fungal triacylglycerol lipase may be selected from lipases of Thermomyces lanuginosa. In one embodiment, at least one Thermomyces lanuginosa lipase is selected from triacylglycerol lipase according to amino acids 1-269 of SEQ ID NO: 2 of U.S. Pat. No. 5,869,438 and variants thereof having lipolytic activity.

Thermomyces lanuginosa lipase may be selected from variants having lipolytic activity which are at least 90%, at least 91%, at least 92%, at least 93%, at least 94%, at least 95%, at least 96%, at least 97%, at least 98% or at least 99% identical when compared to the full length polypeptide sequence of amino acids 1-269 of SEQ ID NO: 2 of U.S. Pat. No. 5,869,438.

Thermomyces lanuginosa lipase may be selected from variants having lipolytic activity comprising conservative mutations only, which do not pertain the functional domain of amino acids 1-269 of SEQ ID NO: 2 of U.S. Pat. No. 5,869,438. Lipase variants of this embodiment having lipolytic activity may be at least 95%, at least 96%, at least 97%, at least 98% or at least 99% similar when compared to the full length polypeptide sequence of amino acids 1-269 of SEQ ID NO: 2 of U.S. Pat. No. 5,869,438.

Thermomyces lanuginosa lipase may be selected from variants having lipolytic activity comprising at least the following amino acid substitutions when compared to amino acids 1-269 of SEQ ID NO: 2 of U.S. Pat. No. 5,869,438: T231R and N233R. Said lipase variants may further comprise one or more of the following amino acid exchanges when compared to amino acids 1-269 of SEQ ID NO: 2 of U.S. Pat. No. 5,869,438: Q4V, V60S, A150G, L227G, P256K.

Thermomyces lanuginosa lipase may be selected from variants having lipolytic activity comprising at least the amino acid substitutions T231R, N233R, Q4V, V60S, A150G, L227G, P256K within the polypeptide sequence of amino acids 1-269 of SEQ ID NO: 2 of U.S. Pat. No. 5,869,438 and are at least 95%, at least 96%, or at least 97% similar when compared to the full length polypeptide sequence of amino acids 1-269 of SEQ ID NO: 2 of U.S. Pat. No. 5,869,438.

Thermomyces lanuginosa lipase may be selected from variants having lipolytic activity comprising the amino acid substitutions T231R and N233R within amino acids 1-269 of SEQ ID NO: 2 of U.S. Pat. No. 5,869,438 and are at least 95%, at least 96%, at least 97%, at least 98%, or at least 99% similar when compared to the full length polypeptide sequence of amino acids 1-269 of SEQ ID NO: 2 of U.S. Pat. No. 5,869,438.

Thermomyces lanuginosa lipase may be a variant of amino acids 1-269 of SEQ ID NO: 2 of U.S. Pat. No. 5,869,438 having lipolytic activity, wherein the variant of amino acids 1-269 of SEQ ID NO: 2 of U.S. Pat. No. 5,869,438 is characterized in containing the amino acid substitutions T231R and N233R.

Thermomyces lanuginosa lipase may be selected from variants having lipolytic activity comprising at least the amino acid substitutions N11K/A18K/G23K/K24A/V77/D130A/V1541/V187T/T189Q or N11K/A18K/G23K/K24A/L75R/V771/D130A/V1541/V187T/T189Q within the polypeptide sequence of amino acids 1-269 of SEQ ID NO: 1 of WO2015/01009 and are at least 95%, at least 96%, or at least 97% similar when compared to the full length polypeptide sequence of amino acids 1-269 of SEQ ID NO: 1 of WO2015/01009.

Lipase variants of this embodiment having lipolytic activity may be at least 60%, at least 70%, at least 75%, at least 80%, at least 85%, at least 90%, at least 91%, at least 92%, at least 93%, at least 94%, at least 95%, at least 96%, at least 97%, at least 98% or at least 99% similar to the full length polypeptide sequence of the parent enzyme.

Preferably, the lipase has at least 60%, at least 65%, at least 70%, at least 75%, at least 80%, at least 85%, at least 90%, at least 95%, at least 98%, at least 99%, or 100% sequence identity with SEQ ID NO: 6 or 7. More preferably, the lipase has at least 80%, at least 85%, at least 90%, at least 95%, at least 98%, at least 99%, or 100% sequence identity with SEQ ID NO: 6 or 7.

### Amylases

“Amylases” according to the invention (alpha and/or beta) include those of bacterial or fungal origin (EC 3.2.1.1 and 3.2.1.2, respectively). Preferably, component (b) comprises at least one enzyme selected from the group of alpha-amylases (EC 3.2.1.1). Chemically modified or protein engineered mutants are included.

Amylases according to the invention have “amylolytic activity” or “amylase activity” involving (endo)hydrolysis of glucosidic linkages in polysaccharides. alpha-amylase activity may be determined by assays for measurement of alpha-amylase activity which are known to those skilled in the art. Examples for assays measuring alpha-amylase activity are:
alpha-amylase activity can be determined by a method employing Phadebas tablets as substrate (Phadebas Amylase Test, supplied by Magle Life Science). Starch is hydrolyzed by the alpha-amylase giving soluble blue fragments. The absorbance of the resulting blue solution, measured spectrophotometrically at 620 nm, is a function of the alpha-amylase activity. The measured absorbance is directly proportional to the specific activity (activity/mg of pure alphaamylase protein) of the alpha-amylase in question under the given set of conditions. Alpha-amylase activity can also be determined by a method employing the Ethyliden-4-nitrophenyl-alpha-D-maltoheptaosid (EPS). D-maltoheptaoside is a blocked oligosaccharide which can be cleaved by an endo-amylase. Following the cleavage, the alpha-glucosidase included in the kit to digest the substrate to liberate a free PNP molecule which has a yellow color and thus can be measured by visible spectophotometry at 405 nm. Kits containing EPS substrate and alpha-glucosidase is manufactured by Roche Costum Biotech (cat. No. 10880078103). The slope of the time dependent absorption-curve is directly proportional to the specific activity (activity per mg enzyme) of the alpha-amylase in question under the given set of conditions.

Amylolytic activity may be provided in units per gram enzyme. For example, 1 unit alpha-amylase may liberate 1.0 mg of maltose from starch in 3 min at pH 6.9 at 20° C.

In one aspect of the present invention, at least one amylase is selected from:
amylases from Bacillus licheniformis having SEQ ID NO:2 as described in WO 95/10603. Suitable variants are described in WO 95/10603 comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Variants are described in WO 94/02597, WO 94/018314, WO 97/043424 and SEQ ID NO:4 of WO 99/019467.
amylases from B. stearothermophilus having SEQ ID NO:6 as disclosed in WO 02/10355 or an amylase with optionally having a C-terminal truncation over the wildtype sequence. Suitable variants of SEQ ID NO:6 include those comprising a deletion in positions 181 and/or 182 and/or a substitution in position 193.
amylases from Bacillus sp. 707 having SEQ ID NO:6 as disclosed in WO 99/19467. Preferred variants of SEQ NO: 6 are those having a substitution, a deletion or an insertion in one or more of the following positions: R¹⁸¹, G182, H183, G184, N195, 1206, E212, E216 and K269.
amylases from Bacillus halmapalus having SEQ ID NO:2 or SEQ ID NO:7 as described in WO 96/23872, also described herein as SP-722. Preferred variants are described in WO 97/3296, WO 99/194671 and WO 2013/001078.
amylases from Bacillus sp. DSM 12649 having SEQ ID NO:4 as disclosed in WO 00/22103.
amylases from Bacillus strain TS-23 having SEQ ID NO:2 as disclosed in WO 2009/061380.
amylases from Cytophaga sp. having SEQ ID NO:1 as disclosed in WO 2013/184577.
amylases from Bacillus megaterium DSM 90 having SEQ ID NO:1 as disclosed in WO 2010/104675.
amylases from Bacillus sp. comprising amino acids 1 to 485 of SEQ ID NO:2 as described in WO 00/60060.
amylases from Bacillus amyloliquefaciens or variants thereof, preferably selected from amylases according to SEQ ID NO: 3 as described in WO 2016/092009.
amylases having SEQ ID NO:12 as described in WO 2006/002643 or amylase variants comprising the substitutions Y295F and M202LITV within said SEQ ID NO:12.
amylases having SEQ ID NO:6 as described in WO 2011/098531 or amylase variants comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] within said SEQ ID NO:6.
amylases having SEQ ID NO:1 as described in WO 2013/001078 or amylase variants comprising an alteration at two or more (several) positions corresponding to positions G304, W140, W189, D134, E260, F262, W284, W347, W439, W469, G476, and G477 within said SEQ ID NO:1.
amylases having SEQ ID NO:2 as described in WO 2013/001087 or amylase variants comprising a deletion of positions 181+182, or 182+183, or 183+184, within said SEM ID NO:2, optionally comprising one or two or more modifications in any of positions corresponding to W140, W159, W167, Q169, W189, E194, N260, F262, W284, F289, G304, G305, R320, W347, W439, W469, G476 and G477 within said SEQ ID NO:2.
amylases which are hybrid alpha-amylases from above mentioned amylases as for example as described in WO 2006/066594;
hybrid amylases according to WO 2014/183920 with A and B domains having at least 90% identity to SEQ ID NO:2 of WO 2014/183920 and a C domain having at east 90% identity to SEQ ID NO:6 of WO 2014/183920, wherein the hybrid amylase has amylolytic activity; preferably the hybrid alpha-amylase is at least 95% identical to SEQ ID NO: 23 of WO 2014/183920 and having amylolytic activity;
hybrid amylase according to WO 2014/183921 with A and B domains having at least 75% identity to SEQ ID NO: 2, SEQ ID NO: 15, SEQ ID NO: 20, SEQ ID NO: 23, SEQ ID NO: 29, SEQ ID NO: 26, SEQ ID NO: 32, and SEQ ID NO: 39 as disclosed in WO 2014/183921 and a C domain having at least 90% identity to SEQ ID NO: 6 of WO 2014/183921, wherein the hybrid amylase has amylolytic activity; preferably, the hybrid alpha-amylase is at least 95% identical to SEQ ID NO: 30 as disclosed in WO 2014/183921 and having amylolytic activity.

Suitable amylases include also those, which are variants of the above described amylases which have amylolytic activity. In one embodiment amylase variants include variants with at least 40 to 100% identity when compared to the full length polypeptide sequence of the parent enzyme as disclosed above. In one embodiment amylase variants having amylolytic activity are at least 70%, at least 75%, at least 80%, at least 85%, at least 90%, at least 91%, at least 92%, at least 93%, at least 94%, at least 95%, at least 96%, at least 97%, at least 98% or at least 99% identical to the full length polypeptide sequence of the parent enzyme as disclosed above. In another embodiment, the invention relates to amylase variants comprising conservative mutations not pertaining the functional domain of the respective amylase. Amylase variants of this embodiment having amylolytic activity may be at least 70%, at least 75%, at least 80%, at least 85%, at least 90%, at least 91%, at least 92%, at least 93%, at least 94%, at least 95%, at least 96%, at least 97%, at least 98% or at least 99% similar to the full length polypeptide sequence of the parent enzyme.

In one embodiment, amylase variants have amylolytic activity according to the present invention when said amylase variants exhibit increased amylolytic activity when compared to the parent amylase.

In one embodiment, amylase variants have amylolytic activity according to the present invention when said amylase variants exhibit at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the amylolytic activity of the respective parent amylase.

In one embodiment, at least one amylase is selected from commercially available amylases which include but are not limited to products sold under the trade names Duramyl™, Termamyl™, Fungamyl™, Stainzyme™, Stainzyme PIus™, Natalase™, Liquozyme X and BAN™ Amplify™, Amplify Prime™ (from Novozymes A/S), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont).

### Mannanases

At least one enzyme comprised in the composition of the invention may be selected from the group of mannan degrading enzyme. At least one mannan degrading enzyme may be selected from β-mannosidase (EC 3.2.1.25), endo-1,4-β-mannosidase (EC 3.2.1.78), and 1,4-β-mannobiosidase (EC 3.2.1.100). Preferably, at least one mannan degrading enzyme is selected from the group of endo-1,4-β-mannosidase (EC 3.2.1.78), a group of enzymes which may be called endo-β-1,4-D-mannanase, β-mannanase, or mannanase herein.

A polypeptide having mannan degrading activity or mannanase activity may be tested for according to standard test procedures known in the art, such as by applying a solution to be tested to 4 mm diameter holes punched out in agar plates containing 0.2% AZCL galactomannan (carob), i.e. substrate for the assay of endo-1,4-beta-D-mannanase available as CatNo. IAZGMA from the company Megazyme (Megazyme's Internet address: http://www.megazyme.com/Purchase/index.html).

Mannan degrading activity may be tested in a liquid assay using carob galactomannan dyed with Remazol Brilliant Bue as described in McCleary, B. V. (1978). Carbohydrate Research, 67(1), 213-221. Another method for testing mannan degrading activity uses detection of reducing sugars when incubated with substrate such as guar gum or locust bean gut—for reference see Miller, G. L. Use of Dinitrosalicylic Acid Reagent for Determination of Reducing Sugars. Analytical Chemistry 1959; 31: 426-428.

At least one mannanase comprised in the composition of the invention may be selected from alkaline mannanase of Family 5 or 26. The term “alkaline mannanase” is meant to encompass mannanases having an enzymatic activity of at least 40% of its maximum activity at a given pH ranging from 7 to 12, preferably 7.5 to 10.5.

At least one mannanase comprised in the composition of the invention may be selected from mannanases originating from Bacillus organisms, such as described in JP-0304706 [beta-mannanase from Bacillus sp.], JP-63056289 [alkaline, thermostable beta-mannanase], JP63036774 [Bacillus microorganism FERM P-8856 producing beta-mannanase and beta-mannosidase at an alkaline pH], JP-08051975 [alkaline beta-mannanases from alkalophilic Bacillus sp. AM-001], WO 97/11164 [mannanase from Bacillus amyloliquefaciens], WO 91/18974 [mannanase active at an extreme pH and temperature], WO 97/11164 [mannanase from Bacillus amyloliquefaciens], WO 2014/100018 [endo-(3-mannanase1 cloned from a Bacillus circulans or Bacillus lentus strain CMG1240 (Bleman1; see U.S. Pat. No. 5,476,775)]. Suitable mannanases are described in WO 99/064619].

At least one mannanase comprised in the composition of the invention may be selected from mannanases originating from Trichoderma organisms, such as disclosed in WO 93/24622. Suitable mannanases include also those, which are variants of the above described mannanases which have mannan degrading activity. In one embodiment mannanase variants include variants with at least 40 to 100% similarity and/or identity when compared to the full length polypeptide sequence of the parent enzyme as disclosed above. In one embodiment mannanase variants having mannan degrading activity are at least 70%, at least 75%, at least 80%, at least 85%, at least 90%, at least 91%, at least 92%, at least 93%, at least 94%, at least 95%, at least 96%, at least 97%, at least 98% or at least 99% similar and/or identical to the full length polypeptide sequence of the parent enzyme as disclosed above.

In one embodiment, mannanase variants have mannan degrading activity according to the present invention when said mannanase variants exhibit increased mannan degrading activity when compared to the parent mannanase.

In one embodiment, mannanase variants have mannan degrading activity according to the present invention when said mannanase variants exhibit at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the mannan degrading activity of the respective parent mannanase.

At least one mannanase may be selected from a commercially available mannanase such as Mannaway® (Novozymes A/S).

### Cellulases

At least one enzyme comprised in the composition of the invention may be selected from the group of cellulases. Cellulases according to the invention include those of bacterial or fungal origin.

At least one cellulase comprised in the composition of the invention may be selected from cellobiohydrolase (1,4-P-D-glucan cellobiohydrolase, EC 3.2.1.91), endo-ss-1,4-glucanase (EC 3.2.1.4) and ss-glucosidase (EC 3.2.1.21). Endoglucanases of EC class 3.2.1.4 may be named endoglucanase, endo-1,4-ss-D-glucan 4-glucano hydrolase, endo-1,4-beta-glucanase, carboxymethyl cellulase, and beta-1,4-glucanase.

Endoglucanases may be classified by amino acid sequence similarities (Henrissat, B. Accessed at UniProt 10/26/2011) under family 5 containing more than 20 endoglucanases of EC 3.2.1.4. Reference is also made to T.-M. Enveri, “Microbial Cellulases” in W. M. Fogarty, Microbial Enzymes and Biotechnology, Applied Science Publishers, p. 183-224 (1983); Methods in Enzymology, (1988) Vol. 160, p. 200-391 (edited by Wood, W. A. and Kellogg, S. T.); Béguin, P., “Molecular Biology of Cellulose Degradation”, Annu. Rev. Microbiol. (1990), Vol. 44, pp. 219248; Begun, P. and Aubert, J-P., “The biological degradation of cellulose”, FEMS Microbiology Reviews 13 (1994) p. 25-58; Henrissat, B., “Cellulases and their interaction with cellulose”, Cellulose (1994), Vol. 1, pp. 169-196.

Preferably, at least one cellulase comprised in the composition of the invention is selected of the glycosyl hydrolase family 7 (GH7, pfam00840), preferably selected from endoglucanases (EC 3.2.1.4).

“Cellulases”, “cellulase enzymes” or “cellulolytic enzymes” according to the invention are enzymes involved in hydrolysis of cellulose. Assays for measurement of “cellulase activity” or “cellulolytic activity” are known to those skilled in the art. For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

Cellulolytic activity may be provided in units per gram enzyme. For example, 1 unit may liberate 1.0 μmole of glucose from cellulose in one hour at pH 5.0 at 37° C. (2 hour incubation time).

In one embodiment, at least one cellulase comprised in the composition of the invention is selected from cellulases comprising a cellulose binding domain. In one embodiment, at least one cellulase is selected from cellulases comprising a catalytic domain only, meaning that the cellulase lacks cellulose binding domain.

In one embodiment, the composition of the invention comprises at least one endoglucanases of EC class 3.2.1.4 is originating from
Bacillus, such as Bacillus sp. CBS 670.93 and CBS 669.93
Melanocarpus, such as Melanocarpus albomyces as disclosed in WO 97/14804
Clostridium, e.g. Clostridium thermocellum
Humicola, such as Humicola insolens (DSM1800) as disclosed in EP 0495257, EP 0531315, EP 0531372, U.S. Pat. Nos. 4,435,307, 5,648,263, 5,776,757, WO 89/09259, WO 91/17244, WO 94/07998 (sequence displayed in FIG. 1 “43 kd human variants thereof), WO 95/24471, WO 96/11262 and WO 98/12307.
Fusarium, such as Fusarium oxysporum e.g. strain J79 (DSM2672) as disclosed in EP 0495257, EP 0531315, EP 0531372, U.S. Pat. Nos. 5,648,263, 5,776,757, WO 89/09259, WO 91/17244, WO 95/24471 and WO 96/11262
Thielavia, such as Thielavia terrestris or Myceliophthora thermophila strain CBS 11765 as disclosed in EP 0531315, U.S. Pat. Nos. 5,648,263, 5,776,757, WO 89/09259, WO 91/17244, WO 95/24471, WO 96/11262, WO 96/29397 (SEQ ID NO: 9 and variants thereof), and WO 98/12307.
Trichoderma, such as Trichoderma reesei, Trichoderma longibrachiatum or Trichoderma harzianum as disclosed in EP 1305432, EP 1240525, WO 92/06165, WO 94/21801, WO 94/26880, WO 95/02043, WO 95/24471 and WO 02/099091.
Aspergillus, such as Aspergillus aculeatus as disclosed in WO 93/17244
Erwinia, such as Erwinia chrysanthermi as described by M. H. Boyer et. al. in European Journal of Biochemistry, vol. 162, page 311-316 (1987).
Acremonium such as Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum as disclosed in WO 96/11262 and WO 96/29397 (SEQ ID NO: 5 and variants thereof).
Cellvibrio such as Cellvibrio mixtus DSM 11683, Cellvibrio mixtus DSM 11684, Cellvibrio mixtus DSM 11685, Cellvibrio mixtus ACM 2601, Cellvibrio mixtus DSM 1523, and Cellvibrio gilvus DSM 11686, as disclosed in WO 98/08940.
Cephalosporium, such as Cephalosporium sp. RYM-202 as disclosed in WO 96/11262.

Suitable cellulases include also those, which are variants of the above described cellulases which have cellulolytic activity. In one embodiment cellulase variants include variants with at least 40 to 100% identity when compared to the full length polypeptide sequence of the parent enzyme as disclosed above. In one embodiment cellulase variants having cellulolytic activity are at least 70%, at least 75%, at least 80%, at least 85%, at least 90%, at least 91%, at least 92%, at least 93%, at least 94%, at least 95%, at least 96%, at least 97%, at least 98% or at least 99% similar and/or identical to the full length polypeptide sequence of the parent enzyme as disclosed above.

In one embodiment, the composition of the invention comprises a Humicola insolens DSM 1800 cellulase complex having endoglucanase, cellobiohydrolase and beta-glucosidase activity.

In one embodiment, the composition of the invention comprises at least one Humicola insolens DSM 1800 endoglucanase (EC 3.2.1.4) having the amino acid sequence disclosed in FIG. 14A-E of WO 91/17244, preferably amino acids 20-434 according said sequence, more preferably having one or more substitutions at positions selected from 182, 223, and 231, most preferably selected from P182S, A223V, and A231V. In one embodiment, the endoglucanase is at least 80% similar and/or identical to a polypeptide according to SEQ ID NO: 2 of WO 95/02675.

In one embodiment, the composition of the invention comprises at least a Bacillus sp. cellulase (EC 3.2.1.4) selected from a polypeptide at least 80% similar and/or identical to the amino acid sequence of position 1 to position 773 of SEQ ID NO: 2 of WO 2004/053039 or a catalytically active fragment thereof.

In one embodiment, the composition of the invention comprises at least a Thielavia terrestris cellulase (EC 3.2.1.4) having a polypeptide at least 80% similar and/or identical to the amino acid sequence of position 1 to position 299 of SEQ ID NO: 4 of WO 2004/053039 or a catalytically active fragment thereof.

In one embodiment, cellulase variants have cellulolytic activity according to the present invention when said cellulase variants exhibit increased cellulolytic activity when compared to the parent cellulase.

In one embodiment, cellulase variants have cellulolytic activity according to the present invention when said cellulase variants exhibit at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the cellulolytic activity of the respective parent cellulase.

At least one cellulase may be selected from Renozyme®, Celluzyme®, Celluclean®, Endolase® and Carezyme® (Novozymes A/S), Clazinase™, and Puradax HA™ (Genencor Int. Inc.), and KAC-500(B)™ (Kao Corporation). At least one peroxidases may be selected from Guardzyme™ (Novozymes A/S).

### DNAses

At least one enzyme comprised in the composition of the invention may be selected from the group of DNA degrading enzymes. Said enzymes usually catalyzes the hydrolytic cleavage of phosphodiester linkages in DNA. The DNAses are classified e.g. in E.C. 3.1.11, E.C. 3.1.12, E.C. 3.1.15, E.C. 3.1.16, E.C. 3.1.21, E.C 3.1.22, E.C 3.1.23, E.C 3.1.24 and E.C.3.1.25 as well as EC 3.1.21.X, where X=1, 2, 3, 4, 5, 6, 7, 8 or 9.

DNAse activity may be determined on DNAse Test Agar with Methyl Green (BD, Franklin Lakes, NJ, USA), which should be prepared according to the manual from supplier. Briefly, 21 g of agar is dissolved in 500 ml water and then autoclaved for 15 min at 121° C. Autoclaved agar is temperated 10 to 48° C. in water bath, and 20 ml of agar is to be poured into petridishes with and allowed to solidify by incubation o/n at room temperature. On solidified agar plates, 5 μl of enzyme solution is added and DNAse activity is observed as colorless zones around the spotted enzyme solutions.

DNAse activity may be determined by using the DNAseAlert™ Kit (11-02-01-04, IDT Intergrated DNA Technologies) according to the supplier's manual. Briefly, 95 μl DNase sample is mixed with 5 μl substrate in a microtiter plate, and fluorescence is immediately measured using e.g. a Clariostar microtiter reader from BMG Labtech (536 nm excitation, 556 nm emission).

At least one DNAse comprised in the composition of the invention may be selected from DNAses originating from Bacillus such as from Bacillus cibi, Bacillus horikoshii, Bacillus horneckiae, Bacillus idriensis, Bacillus algicola, Bacillus vietnamensis, Bacillus hwajinpoensis, Paenibacillus mucilanginosus, Bacillus indicus, Bacillus luciferensis, Bacillus marisflavi; and variants thereof. In one embodiment, at least one DNAse comprised in the composition of the invention is selected from polypeptides 80% identical to SEQ ID NO: 1 of WO 2019/081724. Said polypeptide may comprise one or more substitutions at positions selected from T1, G4, S7, K8, S9, S13, N16, T22, S25, S27, D32, L33, S39, G41, S42, D45, Q48, S57, S59, N61, T65, S66, V76, F78, P91, S101, S106, Q109, A112, S116, T127, S130, T138, Q140, S144, A147, C148, W154, T157, Y159, G162, S167, Q174, G175, L177, S179, and C180—all as disclosed in WO 2019/081724 and WO 2019/081721.

The composition of the invention may comprise DNAse variants having DNA degrading activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar and/or identical when compared to the full-length polypeptide sequence of the corresponding parent enzyme as disclosed above.

According to the present invention, the composition of the invention may comprise a combination of at least two DNAses.

### Other Enzymes

### Acyltransferases/Perhydrolases

At least one enzyme may be selected from acyltransferases (E.C 2.3.1) or perhydrolases. Perhydrolases catalyze perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include acyltransferases with homology to Candida antarctica lipase A (WO 2010/111143) and naturally occurring Mycobacterium perhydrolase enzymes, or variants thereof—e.g. a variant of Mycobacterium smegmatis as described in WO 2005/056782, WO 2008/063400, US 2008145353, and US 2007167344; perhydrolases from the CE7 family (WO 2009/67279), and variants of the M. smegmatis perhydrolase in particular the S54V variant (WO 2010/100028).

### Peroxidases

In order to supply hydrogen peroxide for bleaching purposes in detergent formulations, oxidoreductase enzymes my be employed. The catalyzed reaction is the transfer of electrons from the organic substrate, for the glucose oxidase, for example, from the glucose, to the oxygen as the electron acceptor with the formation of the desired hydrogen peroxide.

“Peroxidase activity” may be measured by the ABTS method as described in Childs et al. 1975 (Biochemical J, 145, p. 93-103) and commercial kits are available from different suppliers. Other measuring methods are known to those known in the art.

The hydrogen peroxide-producing oxidoreductases herein concern enzymes that produce hydrogen peroxide, using oxygen as an electron acceptor. In this regard, particularly preferred oxidoreductases include those of the EC classes E.C. 1.1.3 (CH—OH as the electron donor), E.C. 1.2.3 (aldehyde or oxo groups as the electron donor), E.C. 1.4.3 (CH—NH₂ as the donor), E.C. 1.7.3 (N-containing groups as the donor) and E.C. 1.8.3 (S-containing groups as the donor) come into consideration, wherein enzymes of the EC class EC 1.1.3.

In a preferred embodiment, the hydrogen peroxide-producing oxidoreductase is one in which a sugar is used as the electron donor. The hydrogen peroxide-producing and sugar-oxidizing oxidoreductase is preferably chosen from glucose oxidase (EC 1.1.3.4), hexose oxidase (EC 1.1.3.5), galactose oxidase (EC 1.1.3.9) and pyranose oxidase (EC 1.1.3.10). According to the invention, glucose oxidase (EC 1.1.3.4) is particularly preferred. In one embodiment, aromatic compounds are added that interact with the enzymes to enhance the activity of the oxidoreductases (Enhancer) or to facilitate electron flow (Mediators) between the oxidizing enzymes and the stains over strongly different redox potentials.

At least one enzyme may be selected from oxidases such as amino acid oxidase and polyol oxidase (e.g., WO 2008/051491). Oxidases and their corresponding substrates may be used as hydrogen peroxide generating enzyme systems, and thus a source of hydrogen peroxide. Several enzymes, such as peroxidases, haloperoxidases and perhydrolases, require a source of hydrogen peroxide. By studying EC 1.1.3._, EC 1.2.3._, EC 1.4.3._, and EC 1.5.3._or similar classes (under the International Union of Biochemistry), other examples of such combinations of oxidases and substrates are easily recognized by one skilled in the art.

In one embodiment at least one oxidoreductase is chosen from enzymes that use peroxides as the electron accepter (EC-Classes 1.11 or 1.11.1), in particular, from catalases (EC 1.11.1.6), peroxidases (EC 1.11.1.7), glutathione peroxidases (EC 1.11.1.9), chloride peroxidases (EC 1.11.1.10), manganese peroxidases (EC 1.11.1.13) and/or lignin peroxidases (EC 1.11.1.14), which can also be generally classified under the term peroxidases. Examples of useful peroxidases include peroxidases from Coprinus, e.g. from C. cinereus, and variants thereof as those described in WO 93/24618, WO 95/10602, WO 98/10060 and WO 98/15257.

A peroxidase for use in the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. In one embodiment, the haloperoxidase is a vanadium haloperoxidase, i.e., a vanadate-containing haloperoxidase.

In one embodiment of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g. P. pyrrocinia, and Streptomyces, e.g. S. aureofaciens.

In one embodiment, the haloperoxidase is from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 2001/79459, Dendryphiella salina as described in WO 2001/79458, Phaeotrichoconis crotalarie as described in WO 2001/79461, or Geniculosporium sp. as described in WO 2001/79460.

Commercially available peroxidases include Guardzyme™ (Novozymes A/S), PrimaGreen™ Oxy (DuPont).

### Laccase

At least one enzyme may be selected from laccases. The term “laccase activity” is defined herein as covered by enzyme classification EC 1.10.3.2, or a similar activity, such as a catechol oxidase activity (EC 1.10.3.1), o-aminophenol oxidase activity (EC 1.10.3.4), or bilirubin oxidase activity (EC 1.3.3.5), that catalyzes the oxidation of a substrate using molecular oxygen. “Laccase activity” is determined by oxidation of syringaldazin under aerobic conditions. The violet colour produced is measured at 530 nm. The analytical conditions are 19 μM syringaldazin, 23 mM Tris/maleate buffer, pH 7.5, 30° C., and 1 min reaction time.

Preferred laccase enzymes are enzymes of microbial origin. The enzymes may be derived from plants, bacteria or fungi (including filamentous fungi and yeasts; e.g. Polyporus radiata (WO 92/01046), Coriolus hirsutus (JP 2238885), Coprinopsis cinerea (WO 97/08325), Myceliophthora thermophila (WO 95/33836)).

In one embodiment, laccase is selected from those as described in SEQ ID NO: 2, 4, 6, and 8 of WO 2009/127702 and variants thereof.

At least one laccase may be selected from commercially available laccase Denilite® 1 and 2 from Novozymes.

### Lyase

In one embodiment, at least one enzyme is selected from lyases. “Lyase” may be a pectate lyase derived from Bacillus, particularly B. licheniformis or B. agaradhaerens, or a variant derived of any of these, e.g. as described in U.S. Pat. No. 6,124,127, WO 99/027083, WO 99/027084, WO 2002/006442, WO 2002/092741, WO 2003/095638.

Commercially available pectate lyases are Xpect™, Pectawash™ and Pectaway™ (Novozymes A/S); PrimaGreen™, EcoScour (DuPont).

### Others

In one embodiment, at least one enzyme is selected from the group of pectinases (EC 3.2.1.15 gycosidase), and/or arabinases (EC 3.2.1.99), and/or galactanases (EC 3.2.1.89 and EC 3.2.1.181), and/or xylanases (EC 3.2.1.8, EC 3.2.1.32, EC 3.2.1.136, and EC 3.2.1.156).

In one embodiment, at least one enzyme is a dispersin, preferably at least one dispersin which is at least 80% identical to SEQ ID NO:10 as disclosed in WO2017/186943.

### Compositions

As discussed above, the present invention relates to a composition comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

The composition can be provided in liquid or solid form.

The molar ratio of the protease inhibitor to the enzyme (e.g. subtilisin) is at least 1:1 or 1.5:1, and it is less than 1000:1, more preferred less than 500:1, even more preferred from 100:1 to 2:1 or from 20:1 to 2:1, or most preferred, the molar ratio is from 10:1 to 2:1.

The composition may comprise further enzyme inhibitors or stabilizers. Examples of such enzyme inhibitors or stabilizers are polyols, (e.g., 1,3-propanediol, ethylene glycol, glycerol, and 1,2-propanediol), salts (e.g., CaCl2), MgCl2, NaCl), formic acid, formiate (e.g., sodium formiate), boric acid and boronic acids. Examples of boronic acids are alkyl boronic acids such as methylboronic acid, butylboronic acid, and 2-cyclohexylethylboronic acid; and aryl boronic acids such as phenylboronic acid, 4-methoxyphenylboronic acid, 3,5-dichlorophenylboronic acid, and 4-formylphenylboronic acid (4-FPBA).

Preferably, the composition comprises increased residual activity of the second enzyme different from the protease (a) when compared to a composition comprising a protease not having at least two additional negative charges in the loop region of residues 98 to 104 according to the numbering of SEQ ID NO: 2.

Preferably, the composition comprises at least 70%, at least 80%, or at least 90% residual activity of the second enzyme in presence of the protease (a) compared to the stability of the second enzyme without the protease (a) after 30 days of storage of the composition at 37° C.

Also described herein is a composition, preferably a detergent composition, comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2; and
b) a protease inhibitor selected from the group consisting of peptide aldehyde, peptide aldehyde hydrosulfite adduct, and combinations thereof.

Further described herein is a composition, preferably a detergent composition, comprising
a) a protease
   a1) comprising an amino acid sequence which is at least 80% identical to SEQ ID NO: 1 and
   a2) wherein the protease comprises an amino acid sequence which comprises compared to SEQ ID NO: 1 the amino acid substitution R101E or R101D according to the numbering of SEQ ID NO: 2; and
b) a protease inhibitor selected from the group consisting of peptide aldehyde, peptide aldehyde hydrosulfite adduct, and combinations thereof.

Further described herein is a composition, preferably a detergent composition, comprising
a) a protease
   a1) comprising an amino acid sequence which is at least 80% identical to SEQ ID NO: 1 and
   a2) wherein the protease comprises an amino acid sequence which comprises compared to SEQ ID NO: 1 the amino acid substitution R101E or R101D according to the numbering of SEQ ID NO: 2; and
b) a protease inhibitor being a peptide aldehyde, preferably, wherein the peptide aldehyde is Z-GAY-H or Z-VAL-H.

Further described herein is a composition, preferably a detergent composition, comprising
a) a protease
   a1) comprising an amino acid sequence which is at least 80% identical to SEQ ID NO: 1 and
   a2) wherein the protease comprises an amino acid sequence which comprises compared to SEQ ID NO: 1 the amino acid substitution R101E or R101D according to the numbering of SEQ ID NO: 2 and wherein the protease further comprises the amino acid substitutions S3T, V4I, and V205I according to the numbering of SEQ ID NO: 2; and
b) a protease inhibitor being a peptide aldehyde, preferably, wherein the peptide aldehyde is Z-GAY-H or Z-VAL-H.

### Detergent Compositions

In one aspect the invention therefor relates to a detergent formulation comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, or combinations thereof;
c) at least one second enzyme different from the protease (a); and preferably
d) a surfactant.

The detergent composition may, e.g. be a laundry detergent composition or a dishwashing detergent composition, preferably an automated dishwashing detergent (ADW).

The liquid detergent composition is in a physical form, which is not solid (or gas). It may be a pourable liquid, a pourable gel or a non-pourable gel. It may be either isotropic or structured, preferably it is isotropic. It includes formulations useful for washing in automatic washing machines or for hand washing. The detergent composition contains at least one surfactant. The detergent composition may also include a builder.

The particulate detergent composition may be a granulate or powder, or a powder/granulate pressed to a tablet, briquette. The detergent composition may be in the form of a tablet, bar or pouch, including multi-compartment pouches. The detergent composition can be in the form of a powder, for example a free-flowing powder, such as an agglomerate, spray-dried powder, encapsulate, extrudate, needle, noodle, flake, or any combination thereof.

Pouches can be of any form, shape and material which is suitable for holding the composition, e.g. without allowing the release of the composition from the pouch prior to water contact. The pouch is made from water-soluble film, which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably, the level of polymer in the film for example PVA is at least about 60%. Preferred average molecular weight will typically be about 20,000 to about 150,000. Films can also be a blend compositions comprising hydrolytically degradable and water-soluble polymer blends such as polylactide and polyvinyl alcohol (known under the Trade reference M8630 as sold by Chris Craft In. Prod. Of Gary, Ind., US) plus plasticizers like glycerol, ethylene glycerol, Propylene glycol, sorbitol and mixtures thereof. The pouches can comprise a solid laundry cleaning composition or part components and/or a liquid cleaning composition or part components separated by the water-soluble film. The compartment for liquid components can be different in composition from compartments containing solids (see e.g. US 2009/0011970).

In one embodiment, the composition according to the present invention may be added to a detergent composition in an amount corresponding to 0.001-100 mg of protein, such as 0.01-100 mg of protein, preferably 0.005-50 mg of protein, more preferably 0.01-25 mg of protein, even more preferably 0.05-10 mg of protein, most preferably 0.05-5 mg of protein, and even most preferably 0.01-1 mg of protein per liter of detergent composition.

In a composition such as a liquid or granular detergent, the amount of each enzyme (e.g. subtilisin and optionally a second or more enzymes) will typically be 0.04-80 μM (or μmol/kg), in particular 0.2-30 μM, especially 0.4-20 μM (generally 1-2000 mg/l or mg/kg, in particular 5-750 mg/l, especially 10-500 mg/l) calculated as pure enzyme protein. In a composition such as an enzyme concentrate the amount of each enzyme will typically be 0.01-20 mM, in particular 0.04-10 mM, especially 0.1-5 mM (generally 0.3-500 g/l, in particular 1-300 g/l, especially 3-150 g/l) calculated as pure enzyme protein.

As described above, the detergent composition may comprise one or more surfactants, which may be anionic and/or cationic and/or non-ionic and/or semi-polar and/or zwitterionic, or a mixture thereof. In one embodiment, the detergent composition includes a mixture of one or more non-ionic surfactants and one or more anionic surfactants.

The surfactant(s) is typically present from about 0.1% to 60% by weight, such as from about 1% to about 40%, or from about 3% to about 20%, or from about 3% to about 10%. The surfactant(s) is chosen based on the desired cleaning application, and includes any conventional surfactant(s) known in the art. Any surfactant known in the art for use in detergents may be utilized.

Examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates, including sodium lauryl ether sulfate (SLES), soaps or fatty acids; secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent composition will usually contain from about 1% to about 40% by weight, such as from about 5% to about 30%, including from about 5% to about 15%, or from about 20% to about 25% of an anionic surfactant.

Examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamide (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent composition will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%.

The detergent composition may contain about 0-65% by weight of a builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Examples of builders that can be included are in particular silicates, aluminum silicates (in particular zeolites), carbonates, salts of organic di- and polycarboxylic acids and mixtures of these substances. Non-limiting examples of builders include zeolites, diphosphates (pyrophosphates), triphosphates such as sodium triphosphate (STP or STPP), nitrilotriacetic acid, ethylenediaminetetraacetic acid (EDTA), diethylenetriaminepentaacetic acid, alkyl- or alkenylsuccinic acid, carbonates such as sodium carbonate, soluble silicates such as sodium metasilicate, layered silicates (e.g., SKS-6 from Hoechst), ethanolamines such as 2-aminoethan-1-ol (MEA), iminodiethanol (DEA) and 2,2′,2″-nitrilotriethanol (TEA), and carboxymethylinulin (CMI), and combinations thereof.

The builder may be a strong builder such as methyl glycine diacetic acid (“MGDA”) or N,N-Dicarboxymethyl glutamic acid tetrasodium salt (GLDA); it may be a medium builder such as sodium tri-poly-phosphate (STPP), or it may be a weak builder such as sodium citrate.

Organic builders that can be present in the detergent composition are for example the polycarboxylic acids that can be used in the form of their sodium salts, polycarboxylic acids being understood to be carboxylic acids bearing more than one acid function. These are for example citric acid, adipic acid, succinic acid, glutaric acid, malic acid, tartaric acid, maleic acid, fumaric acid, sugar acids, aminocarboxylic acids, nitrilotriacetic acid (NTA), methyl glycine diacetic acid (MGDA) and derivatives and mixtures thereof. Preferred salts are the salts of polycarboxylic acids such as citric acid, adipic acid, succinic acid, glutaric acid, tartaric acid, sugar acids and mixtures thereof.

Polymeric polycarboxylates are also suitable as builders. These are for example the alkali metal salts of polyacrylic acid or polymethacrylic acid, for example those having a relative molar mass of 600 to 750,000 g/mol.

Suitable polymers are in particular polyacrylates, which preferably have a molar mass of 1000 to 15,000 g/mol. Of this group, owing to their superior solubility, preference can in turn be given to the short-chain polyacrylates having molar masses of 1000 to 10,000 g/mol and particularly preferably of 1000 to 5000 g/mol.

Also suitable are copolymeric polycarboxylates, in particular those of acrylic acid with methacrylic acid and of acrylic acid or methacrylic acid with maleic acid. To improve their solubility the polymers can also contain allyl sulfonic acids, such as allyloxybenzenesulfonic acid and methallyl sulfonic acid, as monomers.

However, soluble builders, such as for example citric acid, or acrylic polymers having a molar mass of 1000 to 5000 g/mol are preferably used.

Within the meaning of this application the molar masses specified for the polymeric polycarboxylates are weight-average molar masses M_{w} of the individual acid form, which were determined in principle by gel permeation chromatography (GPC) using a UV detector. The measurement was carried out against an external polyacrylic acid standard, which because of its structural affinity to the polymers under investigation delivers realistic molar mass values. These figures differ markedly from the molar mass values obtained using polystyrene sulfonic acids as the standard. The molar masses measured against polystyrene sulfonic acids are generally significantly higher than the molar masses given in this publication.

Such organic builder substances can be included if desired in amounts of up to 40 wt.-%, in particular up to 25 wt.-% and preferably from 1 wt.-% to 8 wt.-%. Amounts close to the cited upper limit are preferably used in paste-form or liquid, in particular water-containing, detergent compositions.

In the case that the compositions according to the invention are provided in liquid form, they contain preferably water as the main solvent. Non-aqueous solvents can also or additionally be used. Suitable non-aqueous solvents encompass mono- or polyhydric alcohols, alkanolamines or glycol ethers, provided they are miscible with water in the specified concentration range. The solvents are preferably selected from ethanol, n-propanol, iso-propanol, butanols, glycol, propanediol, butanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol monomethyl ether, dipropylene glycol monoethyl ether, diisopropylene glycol monomethyl ether, diisopropylene glycol monoethyl ether, methoxytriglycol, ethoxytriglycol, butoxytriglycol, 1-butoxyethoxy-2-propanol, 3-methyl-3-methoxybutanol, propylene glycol t-butyl ether, di-n-octyl ether and mixtures of these solvents. It is however preferable for the composition to contain a polyol as the non-aqueous solvent. The polyol can in particular encompass glycerol, 1,2-propanediol, 1,3-propanediol, ethylene glycol, diethylene glycol and/or dipropylene glycol. The composition preferably contains in particular a mixture of a polyol and a monohydric alcohol. Non-aqueous solvents can be used in amounts of between 0.5 and 15 wt-%, but preferably below 12 wt.-%.

To set a desired pH that is not established automatically by mixing the other components, the composition can contain system-compatible and environmentally compatible acids, in particular citric acid, acetic acid, tartaric acid, malic acid, lactic acid, glycolic acid, succinic acid, glutaric acid and/or adipic acid, but also mineral acids, in particular sulfuric acid, or bases, in particular ammonium or alkali hydroxides. Such pH regulators are included in the agents in amounts preferably not exceeding 20 wt.-%, in particular from 1.2 wt.-% to 17 wt.-%.

A composition according to the invention can furthermore contain one or more water-soluble salts, which serve the purpose of viscosity adjustment for example. They can be inorganic and/or organic salts. Inorganic salts that can be used are preferably selected from the group comprising colorless water-soluble halides, sulfates, sulfites, carbonates, hydrogen carbonates, nitrates, nitrites, phosphates and/or oxides of alkali metals, alkaline-earth metals, aluminum and/or transition metals; ammonium salts can also be used. Halides and sulfates of alkali metals are particularly preferred; the inorganic salt is therefore preferably selected from the group comprising sodium chloride, potassium chloride, sodium sulfate, potassium sulfate and mixtures thereof. Organic salts that can be used are for example colorless water-soluble alkali metal, alkaline-earth metal, ammonium, aluminum and/or transition metal salts of carboxylic acids. The salts are preferably selected from the group comprising formate, acetate, propionate, citrate, malate, tartrate, succinate, malonate, oxalate, lactate and mixtures thereof.

The composition can contain one or more thickening agents for thickening purposes. The thickening agent is preferably selected from the group comprising xanthan gum, guar gum, carrageenan, agar agar, gellan, pectin, carob seed meal and mixtures thereof. These compounds are effective thickening agents even in the presence of inorganic salts. The thickening agent additionally stabilizes the continuous, low-surfactant phase and prevents a macroscopic phase separation.

The stability of the enzymes in the detergent composition of the present invention is improved compared to other detergent compositions. Preferably, the composition comprises at least 70%, at least 80%, or at least 90% residual activity of the second enzyme in presence of the protease (a) compared to the stability of the second enzyme without the protease (a) after 30 days of storage of the composition at 37° C.

In turn, also the wash performance of the detergent composition of the present invention is improved compared to other detergent compositions, in particular the enzymatic wash performance, particularly, the lipolytic wash performance, is improved. Preferably, the detergent composition of the present invention is improved in its wash performance, preferably enzymatic, more preferably lipolytic wash performance, by at least 5%, at least 10%, at least 15%, at least 20%, at least 25% or at least 30%.

### METHODS OF USE

The present invention also refers to a method for providing a detergent composition with improved stability and/or wash performance of an enzyme in the detergent composition, wherein the enzyme is not a protease, comprising the use of a detergent composition comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, or combinations thereof;
c) at least one second enzyme different from the protease (a); and
d) a surfactant.

The present invention also refers to the use of a composition comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, or combinations thereof;
c) at least one second enzyme different from the protease (a); and optionally
d) a surfactant

for providing an improved stability and/or wash performance of an enzyme in a detergent composition, wherein the enzyme is not a protease.

Furthermore, the present invention also refers to a method for providing a detergent composition with improved stability and/or wash performance of a second enzyme, which is not a protease, in a detergent composition comprising a peptide aldehyde or a peptide aldehyde hydrosulfite adduct by using a protease comprising an amino acid sequence which is at least 80% identical to SEQ ID NO: 1 and wherein the amino acid sequence of the protease comprises compared to SEQ ID NO: 1 at least two additional negative charges in the loop region of residues 98 to 104 according to the numbering of SEQ ID NO: 2.

### PREFERRED EMBODIMENTS

The present invention is preferably directed to a composition, preferably a detergent composition, comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

A preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

A preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor, wherein the protease inhibitor is a peptide aldehyde, preferably a tripeptide aldehyde, preferably selected from a compound according to formula (IV)

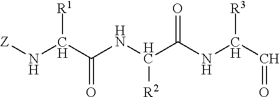

wherein
R¹ and R² are groups such that NH—CHR¹—CO and/or NH—CHR²—CO are non-polar amino acids,
R³ is a group such that NH—CHR³—CO is a non-polar amino acid; and Z is an N-terminal protection group, preferably selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc),
c) at least one second enzyme different from the protease (a), preferably a lipase.

A preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor, wherein the protease inhibitor is a tripeptide aldehyde, preferably selected from a compound according to formula (IV)

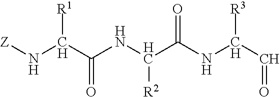

wherein R¹ and R² is a group such that NH—CHR¹—CO and NH—CHR²—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle, preferably independently from each other selected from an L or D-amino acid residue of Ala, Val, Gly and Leu; and
wherein R³ is a group such that NH—CHR³—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle, or wherein R³ is (CH₃)3SiCH₂, preferably independently from each other selected from an L or D-amino acid residue of Tyr, Phe, Val, Ala and Leu; and
wherein Z is an N-terminal protection group, preferably selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc),
c) at least one second enzyme different from the protease (a), preferably a lipase.

A preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor, wherein the protease inhibitor is a tripeptide aldehyde selected from a compound according to formula (IV)

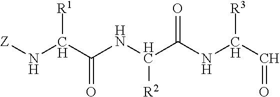

wherein R¹ and R² is a group such that NH—CHR¹—CO and NH—CHR²—CO is an L or D-amino acid residue independently selected from each other of Ala, Val, Gly or Leu; and
wherein R³ is a group such that NH—CHR³—CO is an L or D-amino acid residue of Tyr, Phe, Val, Ala or Leu; and
wherein Z is an N-terminal protection group, preferably selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc),
c) at least one second enzyme different from the protease (a), preferably a lipase.

A preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor, wherein the protease inhibitor is a tripeptide aldehyde selected from a compound according to formula (IV)

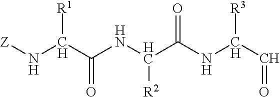

wherein R¹ and R² is a group such that NH—CHR¹—CO and NH—CHR²—CO is an L or D-amino acid residue independently selected from each other of Ala, Val, or Gly, preferably R¹ is Val or Gly and R² is Ala; and
wherein R³ is a group such that NH—CHR³—CO is an L or D-amino acid residue of Tyr or Leu; preferably wherein R³ is Tyr when R¹ is Gly or preferably wherein R³ is Leu when R¹ is Val; and
wherein Z is an N-terminal protection group, preferably selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc),
c) at least one second enzyme different from the protease (a), preferably a lipase.

A preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a), wherein the composition has increased residual activity of the second enzyme when compared to a composition comprising a protease not having the amino acid substitution R101E or R101D according to the numbering of SEQ ID NO: 2.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises compared to SEQ ID NO: 1 the amino acid substitution R101E or R101D, preferably R101E, and the amino acid substitutions S3T, V4I, and V205I according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises compared to SEQ ID NO: 1 the amino acid substitution R101E and the amino acid substitutions S3T, V4I, and V205I according to the numbering of SEQ ID NO: 2;
b) a protease inhibitor, wherein the protease inhibitor is a peptide aldehyde, preferably a tripeptide aldehyde, preferably selected from a compound according to formula (IV)

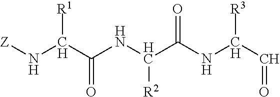

wherein
R¹ and R² are groups such that NH—CHR¹—CO and/or NH—CHR²—CO are non-polar amino acids,
R³ is a group such that NH—CHR³—CO is a non-polar amino acid; and
   Z is an N-terminal protection group, preferably selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc); and
c) at least one second enzyme different from the protease (a), preferably a lipase.

A further preferred embodiment is a composition, preferably a detergent composition, comprising
a) a protease
   a1) comprising an amino acid sequence which is at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a), wherein the composition comprises at least 70%, at least 80%, or at least 90% residual activity of the second enzyme in presence of the protease (a) compared to the stability of the second enzyme without the protease (a) after 30 days of storage of the composition at 37° C.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2, preferably wherein the protease further comprises the amino acid substitutions S3T, V4I, and V205I according to the numbering of SEQ ID NO: 2;
b) a protease inhibitor being a peptide aldehyde, preferably, wherein the peptide aldehyde is Z-GAY-H or Z-VAL-H; and
c) at least one second enzyme different from the protease (a).

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2, and wherein the protease further comprises the amino acid substitutions S3T, V4I, and V205I according to the numbering of SEQ ID NO: 2;
b) a protease inhibitor being Z-VAL-H; and
c) at least one second enzyme different from the protease (a), preferably a lipase.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a), wherein the at least one second enzyme is independently selected from the group consisting of amylase, cellulase, mannanase, lipase, and DNase.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor being a peptide aldehyde; and
c) at least one second enzyme different from the protease (a), wherein the at least one second enzyme is independently selected from the group consisting of amylase, cellulase, mannanase, lipase, and DNase.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor being a tri-peptide aldehyde; and
c) at least one second enzyme different from the protease (a), wherein the at least one second enzyme is independently selected from the group consisting of amylase, cellulase, mannanase, lipase, and DNase.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor being a tri-peptide aldehyde; and
c) at least one second enzyme different from the protease (a), wherein at least one second enzyme is a lipase and wherein the composition comprises at least 70%, at least 80%, or at least 90% residual activity of the second enzyme in presence of the protease (a) compared to the stability of the second enzyme without the protease (a) after 30 days of storage of the composition at 37° C.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2;
b) a protease inhibitor being a tri-peptide aldehyde; and
c) at least one second enzyme different from the protease (a), wherein at least one second enzyme is a lipase.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2; preferably wherein the protease further comprises the amino acid substitutions S3T, V4I, and V205I according to the numbering of SEQ ID NO: 2;
b) a protease inhibitor being the tri-peptide aldehyde Z-GAY-H or Z-VAL-H; and
c) at least one second enzyme different from the protease (a), wherein the at least one second enzyme is independently selected from the group consisting of amylase, cellulase, mannanase, lipase, and DNase, preferably, the second enzyme being a lipase.

A further preferred embodiment is a composition, preferably a detergent composition, comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E according to the numbering of SEQ ID NO: 2; and the amino acid substitutions S3T, V4I, and V205I according to the numbering of SEQ ID NO: 2;
b) a protease inhibitor being the tri-peptide aldehyde Z-GAY-H or Z-VAL-H, preferably Z-VALH; and
c) at least one second enzyme different from the protease (a), wherein the at least one second enzyme is independently selected from the group consisting of amylase, cellulase, mannanase, lipase, and DNase, preferably, the second enzyme being a lipase.

A further preferred embodiment is a method for providing a detergent composition with improved stability and/or wash performance of an enzyme in the detergent composition, wherein the enzyme is not a protease, comprising the use of a detergent composition comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

A further preferred embodiment is a method for providing a detergent composition with improved stability and/or wash performance of an enzyme in the detergent composition, wherein the enzyme is not a protease, comprising the use of a detergent composition comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2; preferably wherein the protease further comprises the amino acid substitutions S3T, V4I, and V205I according to the numbering of SEQ ID NO: 2;
b) a protease inhibitor being the tri-peptide aldehyde Z-GAY-H or Z-VAL-H; and
c) at least one second enzyme different from the protease (a), wherein the at least one second enzyme is independently selected from the group consisting of amylase, cellulase, mannanase, lipase, and DNase, preferably, the second enzyme being a lipase.

A further preferred embodiment is the use of a composition comprising
a) a protease
   a1) comprising an amino acid sequence which is at least 80% identical to SEQ ID NO: 1 and
   a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a) for providing a detergent composition with improved stability and/or wash performance of an enzyme in the detergent composition, wherein the enzyme is not a protease.

A further preferred embodiment is the use of a composition comprising
a) a protease
   a1) comprising an amino acid sequence which is at least 80%, at least 85%, at least 90%, at least 95%, at least 96%, at least 97%, at least 98%, at least 99%, or 100% identical to SEQ ID NO: 1 and
   a2) wherein the protease comprises an amino acid sequence which comprises compared to SEQ ID NO: 1 the amino acid substitution R101E or R101D, preferably R101E, according to the numbering of SEQ ID NO: 2; preferably wherein the protease further comprises the amino acid substitutions S3T, V4I, and V205I according to the numbering of SEQ ID NO: 2;
b) a protease inhibitor being the tri-peptide aldehyde Z-GAY-H or Z-VAL-H; and
c) at least one second enzyme different from the protease (a), wherein the at least one second enzyme is independently selected from the group consisting of amylase, cellulase, mannanase, lipase, and DNase, preferably, the second enzyme being a lipase, for providing a detergent composition with improved stability and/or wash performance of an enzyme in the detergent composition, wherein the enzyme is not a protease.

### Examples

General experimental details: Residual activities of protease and lipase enzymes after different storage times are measured using standard analytical methods. Protease activity was measured by hydrolysis of N,N-Dimethylcasein at 50° C., pH 9.5 and lipase was measured using pNP-Valerate at 30° C., pH8.0.

### Example 1

### Preservation of Lipase (According to SEQ ID NO: 6) Activity

For analyzing the residual enzyme stability protease, lipase and protease inhibitor was dosed in detergent ES1 (Maranil DBS/LC LAS 5.5% w/w, Edenor coco fatty acid C12-C18 coco fatty acid 2.4% w/w, Lutensol AO7 AEO 5.4% w/w, Texapon N70 FAEO 5.4% w/w, 1,2 propylene glycol 6.0% w/w, ethanol 2.0% w/w, KOH 2.2% w/w) plus 3% sodium citrate resulting in an pH of 8.0-8.5. The protease inhibitor was always added in the 3-fold molar excess to the protease.

Following formulations were created, with all the proteases being dosed in the formulation at a level of 15 μM and the protease inhibitor 45 μM, respectively. As protease inhibitor Z-VAL-H was used. The lipase (SEQ ID NO: 6) was dosed at a concentration of 1.4 μM:

| Formulation | 1 | 2 | 3 | 4 | 5 |
|---|---|---|---|---|---|
| Protease | SEQ | SEQ | SEQ | SEQ | − |
| | ID NO: | ID NO: | ID NO: | ID NO: | |
| | 5 | 4 | 5 | 4 | |
| Lipase | + | + | + | + | + |
| SEQ ID | | | | | |
| NO: 6 | | | | | |
| Z-VAL-H | + | + | − | − | − |

The formulations are stored for 30 days at 37° C. and aliquots were taken for residual enzyme determination after 1 d, 8 d, 14 d and 30 d. Prior measurement the samples were kept frozen at −20° C.

The results for the residual lipase activity are shown in following table with the value at day 0 being 100%:

| Formulation | | 1 | 2 | 3 | 4 | 5 |
|---|---|---|---|---|---|---|
| 0 | d | 100% | 100% | 100% | 100% | 100% |
| 8 | d | 22% | 72% | 0% | 1% | 85% |
| 14 | d | 8% | 47% | 0% | 0% | 54% |
| 30 | d | 0% | 15% | 0% | 0% | 29% |

The results for the residual protease activity are shown in following table with the value at day 0 being 100%:

| | | | | | |
|---|---|---|---|---|---|
| Formulation | 1 | 2 | 3 | 4 | 5 |

| | | | | | | |
|---|---|---|---|---|---|---|
| 0 | d | 100% | 100% | 100% | 100% | — |
| 8 | d | 81% | n.a. | 22% | 46% | — |
| 14 | d | 78% | 65% | 0% | 17& | — |
| 30 | d | 67% | 61% | 0% | 0% | — |

The protease is as expected stabilized by the protease inhibitor, but the stabilized proteases show similar residual activities independent of the charge in the active site loop.

It is visible that the addition of the protease inhibitor greatly increases the residual activity of the lipase SEQ ID NO: 6 especially in combination with variants having an introduction of 2 negative charges in the active site loop (Formulation 2 with SEQ ID NO: 4 does contain a R101E mutation with the numbering according to SEQ ID NO: 2).

Surprising is the effect on the lipase as the stabilizing effect of the protease inhibitor on the protease only is not improved in proteases having two negative charges introduced in the active site loop.

### Example 2

### Preservation of the Lipase (According to SEQ ID NO: 7) Activity

For analyzing the residual enzyme stability protease, lipase and protease inhibitor was dosed in detergent ES1 (Maranil DBS/LC LAS 5.5% w/w, Edenor coco fatty acid C12-C18 coco fatty acid 2.4% w/w, Lutensol A07 AEO 5.4% w/w, Texapon N70 FAEO 5.4% w/w, 1,2 propylene glycol 6.0% w/w, ethanol 2.0% w/w, KOH 2.2% w/w) plus 3% sodium citrate resulting in an pH of 8.0-8.5. The protease inhibitor was always added in the 3-fold molar excess to the protease.

Following formulations were created, with all the proteases being dosed in the formulation at a level of 15 μM and the protease inhibitor 45 μM, respectively. As protease inhibitor Z-VAL-H was used. The lipase (SEQ ID NO: 7) was dosed at a concentration of 1.4 μM.

| Formulation | 1 | 2 | 3 | 4 | 5 |
|---|---|---|---|---|---|
| Protease | SEQ | SEQ | SEQ | SEQ | − |
| | ID NO: | ID NO: | ID NO: | ID NO: | |
| | 5 | 4 | 5 | 4 | |
| Lipase SEQ | + | + | + | + | + |
| ID NO: 7 | | | | | |
| Z-VAL-H | + | + | − | − | − |

The formulations are stored for 30 days at 37° C. and aliquots were taken for residual enzyme determination after 1 d, 8 d, 14 d and 30 d. Prior measurement the samples were kept frozen at −20° C.

The results for the residual lipase activity are shown in following table with the value at day 0 being 100%:

| Formulation | | 1 | 2 | 3 | 4 | 5 |
|---|---|---|---|---|---|---|
| 0 | d | 100% | 100% | 100% | 100% | 100% |
| 8 | d | 51% | 81% | 0% | 64% | 65% |
| 14 | d | 39% | 61% | 0% | 36% | 53% |
| 30 | d | 11% | 37% | 0% | 9% | 30% |

The results for the residual protease activity are shown in following table with the value at day 0 being 100%:

| | | | | | |
|---|---|---|---|---|---|
| Formulation | 1 | 2 | 3 | 4 | 5 |

| | | | | | | |
|---|---|---|---|---|---|---|
| 0 | d | 100% | 100% | 100% | 100% | — |
| 8 | d | 74% | 100% | 21% | 39% | — |
| 14 | d | 69% | 76% | 12% | 29& | — |
| 30 | d | 61% | 68% | 0% | 0% | — |

The protease is as expected stabilized by the protease inhibitor, but the stabilized proteases do have similar stability independent of the charge in the active site loop.

It is visible that the addition of the protease inhibitor greatly increases the residual activity of the lipase SEQ ID NO: 7 especially in combination with variants having an introduction of 2 negative charges in the active site loop (Formulation 2 with SEQ ID NO: 4 does contain a R101E mutation with the numbering according SEQ ID NO: 2). The stabilization is in the magnitude of the absence of the protease (Formulation 2 versus Formulation 5). Surprising is the effect on the lipase as the stabilization of the protease only is not visible for proteases having two negative charges introduced in the active site loop.

1. A composition comprising
a) a protease
a1) comprising an amino acid sequence which is at least 80% identical to SEQ ID NO: 1 and
a2) wherein the amino acid sequence of the protease comprises compared to SEQ ID NO: 1 at least two additional negative charges in the loop region of residues 98 to 104 according to the numbering of SEQ ID NO: 2;
b) a protease inhibitor selected from the group consisting of peptide aldehyde, peptide aldehyde hydrosulfite adduct, and combinations thereof; and
c) at least one second enzyme different from the protease (a).

2. The composition of claim 1, wherein the protease comprises an amino acid sequence which comprises compared to SEQ ID NO: 1 the amino acid substitution R101E or R101D according to the numbering of SEQ ID NO: 2.

3. The composition of claim 1, wherein the protease according to the numbering of SEQ ID NO: 2 comprises at least one of the amino acid residues selected from the group consisting of
a. threonine or serine at position 3 (3T or 3S),
b. isoleucine or valine at position 4 (41 or 4V),
c. serine, alanine, threonine or arginine at position 63 (63S, 63A, 63T or 63R),
d. threonine, aspartic acid or glutamic acid at position 156 (156T, 156D, or 156E),
e. serine or proline at position 194 (194S or 194P),
f. serine, valine, or methionine at position 199 (199S, 199V, or 199M)
g. isoleucine or valine at position 205 (205I or 205V); and
h. aspartic acid, glutamic acid, glutamine, glycine at position or leucine at position 217 (217D, 217E, 217Q, 217G or 217L).

4. The composition of claim 1, wherein the protease comprises compared to SEQ ID NO: 1 the amino acid substitution R101E or R101D and the amino acid substitutions S3T, V4I, and V205I according to the numbering of SEQ ID NO: 2.

5. The composition of a claim 1, wherein the protease comprises compared to SEQ ID NO: 1 the amino acid substitution R101 E or R101 D and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T,D and L262N,Q,D according to the numbering of SEQ ID NO: 2.

6. The composition of claim 1, wherein the protease has an additional mutation at position 217 according to the numbering of SEQ ID NO: 2.

7. The composition of claim 1, wherein the protease comprises an amino acid sequence selected from the group consisting of
a) amino acid sequence of SEQ ID NO: 3,
b) amino acid sequence of SEQ ID NO: 3, wherein the amino acid sequence comprises at least one additional amino acid substitution selected from the group consisting of
i. threonine at position 3 (3T);
ii. isoleucine at position 4 (41);
iii. serine, alanine, threonine or arginine at position 63 (63S, 63A, 63T, or 63R);
iv. threonine, aspartic acid or glutamic acid at position 156 (156T, 156D, or 156E);
v. serine or proline at position 194 (194S or 194P);
vi. methionine or serine at position 199 (199M or 199S);
vii. isoleucine at position 205 (205I); and
viii. aspartic acid, glutamic acid, glutamine or glycine at position 217 (217D, 217E, 217Q or 217G);
c) amino acid sequence of SEQ ID NO: 4, and
d) amino acid sequence of SEQ ID NO: 4, wherein the amino acid sequence comprises at least one additional amino acid substitution selected from the group consisting of
i. serine at position 3 (3S);
ii. valine at position 4 (4V);
iii. serine, alanine, threonine or arginine at position 63 (63S, 63A, 63T, or 63R);
iv. threonine, aspartic acid or glutamic acid at position 156 (156T, 156D, or 156E);
v. serine or proline at position 194 (194S or 194P);
vi. methionine or serine at position 199 (199M or 199S);
vii. valine at position 205 (205V); and
viii. aspartic acid, glutamic acid, glutamine or glycine at position 217 (217D, 217E, 217Q or 217G).

8. The composition of claim 1, wherein the protease inhibitor is a peptide aldehyde.

9. (canceled)

10. (canceled)

11. The composition of claim 1, wherein the composition further comprises a surfactant.

12. The composition of claim 1, wherein the at least one second enzyme different from the protease (a) is independently selected from the group consisting of oxidoreductase, transferase, hydrolase, lyase, isomerase, ligase, aminopeptidase, amylase, asparaginase, carbohydrase, carboxypeptidase, catalase, cellulase, chitinase, cutinase, cyclodextrin glycosyltransferase, deoxyribonuclease, esterase, alpha-galactosidase, betagalactosidase, glucoamylase, alpha-glucosidase, beta-glucosidase, hyaluronic acid synthase, invertase, laccase, lipase, mannosidase, mutanase, oxidase, a pectinolytic enzyme, peroxidase, phytase, polyphenoloxidase, protease, ribonuclease, transglutaminase, dispersin, and orxylanase.

13. Detergent composition comprising the composition according to claim 11.

14. Detergent composition of claim 13, wherein the detergent composition is a dishwashing detergent composition or a laundry detergent composition.

15. Method for providing a detergent composition with improved stability and/or wash performance of an enzyme in the detergent composition, wherein the enzyme is not a protease, comprising the use of a detergent composition according to claim 1.

16. (canceled)

17. The composition of claim 8, wherein the protease inhibitor is selected from a compound according to formula (IV) 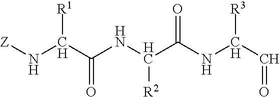
wherein
R¹ and R² are groups such that NH—CHR¹—CO and/or NH—CHR²—CO are non-polar amino acids,
R³ is a group such that NH—CHR³—CO is a non-polar amino acid; and
Z is an N-terminal protection group.

18. The composition of claim 17, wherein Z is an N-terminal protection group selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc).

19. The composition of claim 6, wherein the additional mutation at position 217 according to the numbering of SEQ ID NO: 2 is L217Q, L217D, L217E, or L217G.

20. The composition of claim 17, wherein R¹ and R² is a group such that NH—CHR¹—CO and NH—CHR²—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle.

21. The composition of claim 17, wherein R³ is a group such that NH—CHR³—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle, or wherein R³ is (CH₃)₃SiCH₂.
